SEQUENCE LISTING (8 sequences)

SEQ ID NO: 1 - protein, 320 aa, synthetic construct
  REGION 1..320: note = Synthetic
  LIPID 1: note = N-palmitoyl-S-(2RS)-2,3-bis-(palmitoyloxy) propyl cysteine
CSSFNEKGEVSEKIITRADGTRLEYTGIKSDGSGKAKEVLKNFTLEGKVANDKTTLVVKCGTVTLSKNISKSGEVSVELNDTDSSAATKKTAAWNSGTSTLTITVNSKKTKDLVFTKENTITVQQYDSNGTKLEGSAVEITKLDEICNALKGTSDKNNGSGSKEKNKDGKYSFNEKGELSAKTMTRENGTKLEYTEMKSDGTGKAKEVLKNFTLEGKVANDKVTLEVKCGTVTLSKEIAKSGEVTVALNDTNTTQATKKTGAWDSKTSTLTISVNSKKTTQLVFTKQDTITVQKYDSAGTNLEGTAVEIKTLDELCNALK

SEQ ID NO: 2 - protein, 322 aa, synthetic construct
  REGION 1..322: note = Synthetic
  LIPID 1: note = N-palmitoyl-S-(2RS)-2,3-bis-(palmitoyloxy) propyl cysteine
CSSFNAKGELSEKTILRANGTRLEYTEIKSDGTGKAKEVLKDFALEGTLAADKTTLKVTCGTVVLSKHIPNSGEITVELNDSNSTQATKKTGKWDSNTSTLTISVNSKKTKNIVFTKEDTITVQKYDSAGTNLEGNAVEIKTLDELCNALKGTSDKNNGSGSKEKNKDGKYSFNEKGEVSEKILTRSNGTTLEYSQMTDAENATKAVETLKNGIKLPGNLVGGKTKLTVTCGTVTLSKNISKSGEITVALNDTETTPADKKTGEWKSDTSTLTISKNSQKTKQLVFTKENTITVQNYNRAGNALEGSPAEIKDLAELCAALK

SEQ ID NO: 3 - protein, 321 aa, synthetic construct
  REGION 1..321: note = Synthetic
  LIPID 1: note = N-palmitoyl-S-(2RS)-2,3-bis-(palmitoyloxy) propyl cysteine
CSSFNEKGEISEKTIVRANGTRLEYTDIKSDKTGKAKEVLKDFTLEGTLAADGKTTLKVTCGTVTLSKNISKSGEITVALDDTDSSGNKKSGTWDSGTSTLTISKNRTKTKQLVFTKEDTITVQNYDSAGTNLEGKAVEITTLKELCNALKGTSDKNNGSGSKEKNKDGKYSFNGKGETSEKTIVRANGTRLEYTDIKSDGSGKAKEVLKDFTLEGTLAADGKTTLKVTCGTVVLSKNILKSGEITAALDDSDTTRATKKTGKWDSKTSTLTISVNSQKTKNLVFTKEDTITVQRYDSAGTNLEGKAVEITTLKELCNALK

SEQ ID NO: 4 - DNA, 1020 bp, synthetic construct
  misc_feature 1..1020: note = Synthetic
  source 1..1020: mol_type = other DNA
atgaaagctactaaactggtactgggcgcggtaatcctgggttctactctgctggcaggttgctcaagcttcaacgaaaagggcgaagtcagcgaaaaaatcattacccgcgcagacggcacccgcctggaatacaccggcatcaaatcggacggcagcggcaaagcgaaagaagttctgaaaaactttaccctggaaggcaaagtcgcaaatgataaaaccaccctggtggtgaaatgcggcaccgttacgctgagcaaaaacattagtaaatccggtgaagtctctgtggaactgaatgataccgacagctctgcggccaccaagaaaaccgcagcttggaactcaggcacctcgacgctgaccattacggttaatagcaagaaaaccaaagatctggtcttcacgaaagaaaacaccatcacggtgcagcaatatgacagcaatggtaccaaactggaaggctccgctgtggaaatcacgaaactggatgaaatctgtaatgctctgaaaggtactagtgacaaaaacaatggctctggtagcaaagagaaaaacaaagatggcaagtactcattcaacgaaaaaggcgaactgtcggcgaaaacgatgacgcgtgaaaacggcaccaaactggaatatacggaaatgaaaagcgatggcaccggtaaagcgaaagaagttctgaaaaactttaccctggaaggcaaagtcgccaatgacaaagtcaccctggaagtgaaatgcggcaccgttacgctgtcaaaagaaattgcaaaatcgggtgaagtgaccgttgctctgaacgatacgaataccacgcaagcgaccaagaaaaccggcgcctgggacagcaaaacctctacgctgaccattagtgttaatagcaagaaaaccacgcagctggtcttcaccaaacaagatacgatcaccgtgcagaaatacgacagtgcgggtaccaacctggaaggcacggctgttgaaatcaaaaccctggacgaactgtgtaacgccctgaaa

SEQ ID NO: 5 - DNA, 1029 bp, synthetic construct
  misc_feature 1..1029: note = Synthetic
  source 1..1029: mol_type = other DNA
atgaaagctactaaactggtactgggcgcggtaatcctgggttctactctgctggcaggttgctcaagcttcaatgctaagggcgaactgagcgaaaaaacgatcctgcgtgcgaatggcacccgtctggaatacaccgaaatcaaatccgatggtacgggcaaagcaaaggaagtcctgaaagattttgctctggaaggtaccctggcggccgacaaaaccacgctgaaggtgacgtgcggcaccgtggttctgagcaaacatattccgaactctggtgaaatcaccgttgaactgaacgatagcaattctacgcaggcaaccaaaaagacgggcaaatgggacagtaatacctccacgctgaccatttcagtcaactcgaaaaagaccaaaaatattgtgttcacgaaggaagatacgatcaccgttcaaaaatatgactccgcgggcaccaacctggaaggcaatgccgtcgaaatcaaaaccctggatgaactgtgtaacgccctgaagggtactagtgacaaaaacaatggctctggtagcaaagagaaaaacaaagatggcaagtactcattcaacgaaaaaggcgaagtgagcgaaaaaattctgacccgtagcaatggcaccaccctggaatatagccagatgaccgatgcagaaaatgcaaccaaagcagttgaaaccctgaaaaacggtattaaactgcctggtaatctggttggtggtaaaaccaaactgaccgttacctgtggcaccgttaccctgagcaaaaacattagcaaaagcggtgaaattaccgtggcactgaatgataccgaaaccacaccggcagacaaaaaaaccggtgaatggaaaagcgataccagcaccctgaccattagtaaaaatagccagaaaacaaaacagctggtgtttaccaaagaaaacaccattaccgtgcagaattataaccgtgcaggtaatgcactggaaggtagtccggcagaaattaaagatctggcagaactgtgtgcagccctgaaataa

SEQ ID NO: 6 - DNA, 1023 bp, synthetic construct
  misc_feature 1..1023: note = Synthetic
  source 1..1023: mol_type = other DNA
atgaaagctactaaactggtactgggcgcggtaatcctgggttctactctgctggcaggttgctcaagcttcaacgaaaagggcgaaatctcagaaaaaaccatcgtccgcgctaacggcacccgcctggaatacaccgacatcaaatcagacaagaccggtaaagcgaaggaagttctgaaagattttacgctggaaggtaccctggcagcagacggtaaaaccacgctgaaggtgacctgcggtaccgttacgctgtccaaaaacattagtaagtccggcgaaatcacggtcgccctggatgacaccgatagctctggcaacaaaaagagcggtacctgggattcaggcacctcgacgctgaccatttctaaaaatcgtacgaaaaccaagcagctggtcttcacgaaagaagatacgatcaccgtgcaaaactatgacagcgcaggtaccaatctggaaggcaaagctgtggaaattaccacgctgaaagaactgtgtaatgctctgaaaggtactagtgacaaaaacaatggctctggtagcaaagagaaaaacaaagatggcaagtactcattcaacggcaaaggtgaaacgagcgaaaagaccatcgtgcgtgcgaacggtacccgcctggaatatacggacattaaatcggacggcagcggcaaagcaaaggaagtcctgaaagattttacgctggaaggtaccctggcagcagacggtaaaaccacgctgaaggtgacgtgcggcaccgtggttctgtcaaaaaacattctgaagtcgggtgaaatcaccgcagctctggatgacagcgataccacgcgtgctacgaaaaagaccggtaaatgggatagcaagacctctacgctgaccattagtgtcaactcccagaaaacgaagaatctggtgttcaccaaagaagatacgatcaccgttcaacgctatgacagtgcgggcaccaacctggaaggcaaagccgttgaaattaccacgctgaaagaactgtgtaatgctctgaaa

SEQ ID NO: 7 - protein, 21 aa, synthetic construct
  REGION 1..21: note = Synthetic
GTSDKNNGSGSKEKNKDGKYS

SEQ ID NO: 8 - protein, 11 aa, synthetic construct
  REGION 1..11: note = Synthetic
NFTLEGKVAND

(19) United States
(12) Patent Application Publication
(10) Pub. No.: US 2026/0295025 A1
(43) Pub. Date: Oct. 1, 2026

(54) COMPOSITIONS COMPRISING THREE OSPA FUSION PROTEINS FOR MEDICAL USE

(71) Applicants: Valneva Austria GmbH, Vienna (AT); Pfizer Inc., New York, NY (US)

(72) Inventors: Nicole Bézay, Hinterbrühl (AT); Romana Hochreiter, Wien (AT); Urban Lundberg, Pressbaum (AT); Steven Russell Bailey, Bryn Mawr, PA (US); Annaliesa Sybil Anderson, Upper Saddle River, NJ (US); Kathrin Ute Jansen, New York, NY (US); Daniel Alfred Scott, Spring City, PA (US)

(73) Assignees: Valneva Austria GmbH, Vienna (AT); Pfizer Inc., New York, NY (US)

(21) Appl. No.: 19/401,907

(22) Filed: Nov. 26, 2025

Related U.S. Application Data
(63) Continuation of application No. 17/917,624, filed on Apr. 9, 2021, now Pat. No. 12,514,912.
(60) Provisional application No. 63/152,582, filed on Feb. 23, 2021.

(30) Foreign Application Priority Data
Apr. 9, 2020 (EP) 20169147.4
Apr. 9, 2020 (EP) 20169148.2
Apr. 15, 2020 (EP) 20169642.4
Jul. 22, 2020 (EP) 20187283.5
Oct. 20, 2020 (EP) 20202816.3

(51) Int. Cl. A61K 39/02; A61K 39/00; A61P 37/04

(52) U.S. Cl. CPC A61K 39/0225; A61P 37/04; A61K 2039/545; A61K 2039/55505

(57) ABSTRACT

The present invention relates to a composition comprising the OspA fusion protein of SEQ ID NO: 1 (LipS1D1-S2D1), the OspA fusion protein of SEQ ID NO: 2 (Lip-S4D1-SShybD1) and the OspA fusion protein of SEQ ID NO: 3 (Lip-S5D1-S6D1) for use in a vaccine or for use in a method for eliciting an immune response in a human against Lyme disease.

### RELATED APPLICATIONS

This application is a continuation of U.S. application Ser. No. 17/917,624, filed Oct. 7, 2022, which is a national stage filing under 35 U.S.C. § 371 of International Patent Application Serial No. PCT/US2021/026599, filed Apr. 9, 2021, which claims priority to and the benefit of U.S. Provisional Application Ser. No. 63/152,582, filed Feb. 23, 2021. The entire contents of these applications are incorporated herein by reference in their entirety.

### REFERENCE TO AN ELECTRONIC SEQUENCE LISTING

The contents of the electronic sequence listing (1042270135US02-SEQ-NTJ.xml; Size: 16,215 bytes and Date of Creation: Nov. 26, 2025) are herein incorporated by reference in their entirety.

### FIELD OF THE INVENTION

The present invention relates to a composition comprising the OspA fusion protein of SEQ ID NO: 1 (LipS1D1-S2D1), the OspA fusion protein of SEQ ID NO: 2 (Lip-S4D1-S3hybD1) and the OspA fusion protein of SEQ ID NO: 3 (Lip-S5D1-S6D1) for use in a vaccine or for use in a method for eliciting an immune response in a human against Lyme disease.

### BACKGROUND OF THE INVENTION

Lyme borreliosis (herein also referred to as “LB”), also called Lyme disease (herein also referred to as “LD”), is an emerging, tick-borne zoonotic disease caused by several genospecies of the spirochete Borrelia burgdorferi sensu lato (s.l.). It is the most common vector-borne disease in Europe and North America [Mead P. S., Epidemiology of Lyme Disease, Infect Dis Clin N Am 29 (2015) 187-210]. In Europe, 65,000-85,000 cases are notified per year, however, due to inconsistent case reporting and the fact that LB is often undiagnosed, this number is certainly an underestimation [Rizzoli A, et al. Lyme borreliosis in Europe. Euro Surveill 16. (2011); Lindgren E, Jaenson T G T, Organization WHO Europe (2006) Lyme borreliosis in Europe: Influences of climate and climate change, epidemiology, ecology and adaptation measures, euro.who.int/data/assets/pdf_file/0006/96819/E89522.pdf, last assessed March 2016]. The US Centers for Disease Control and Prevention (CDC) estimates about 300,000 cases annually in the United States, a number which is nearly 10-fold higher than reported cases [Hinckley A F, et al. Lyme disease testing by large commercial laboratories in the United States. Clin Infect Dis. 2014 Sep. 1; 59 (5): 676-81. doi: 10.1093/cid/ciu397. Epub 2014 May 30; Nelson C A, et al. Incidence of Clinician-Diagnosed Lyme Disease, United States, 2005-2010. Emerg Infect Dis. 2015 September; 21 (9): 1625-31. doi: 10.3201/eid2109.150417]. The incidence of LB has a bimodal distribution with respect to age. The two main target populations are children aged 5-14 years and adults aged 50-64 years [Rizzoli A, et al. 2011, supra; cdc.gov/lyme/stats/graphs.html, last assessed March 2016].

In Europe, most human infections are caused by four Borrelia genospecies, presenting six OspA serotypes (STs): B. afzelii (ST2), B. garinii (ST3, ST5 and ST6), B. burgdorferi sensu stricto (s.s.) (ST1) and B. bavariensis (ST4). In the US, B. burgdorferi s.s. (ST1) is found in almost 100% of cases. Recently, a new genospecies named Borrelia mayonii has been described, which was found in few clinical specimen isolated in the Upper Midwest of the US [Pritt B S, et al. Identification of a novel pathogenic Borrelia species causing Lyme borreliosis with unusually high spirochaetaemia: a descriptive study, Lancet Infect Dis 2016; 16:556-64].

The most common clinical manifestation of LD is a gradually expanding erythematous skin rash called erythema migrans (EM), a distinct sign of early localized Borrelia infection. An EM appears within days to weeks at the location of the tick bite and is often accompanied by symptoms of fatigue, fever, headache, mild stiff neck, arthralgia, or myalgia [Mead et al., 2015, supra]. In approximately 70%-80% of LD cases, patients develop EM [Stanek G, et al. Lyme borreliosis, Lancet 2012; 379:461-73; Wormser G P, et al, The Clinical Assessment, Treatment, and Prevention of Lyme Disease, Human Granulocytic Anaplasmosis, and Babesiosis: Clinical Practice Guidelines by the Infectious Diseases Society of America, Clinical Infectious Diseases 2006; 43:1089-134.]

If untreated or treated inadequately with antibiotics, the infection can disseminate to other parts of the body and can cause serious late-stage manifestations affecting the nervous system (facial palsy, meningitis, myelitis, and encephalitis), joints (recurrent or persistent large joint synovitis), or heart (e.g. conduction abnormalities and carditis).

The most common late stage clinical manifestations of LB that develops in about 30% of patients include musculoskeletal manifestations, such as Lyme arthritis. Lyme arthritis comprises recurrent attacks or long-lasting joint swelling (synovitis), usually in one or a few joints most commonly the knee, which develops months after a tick bite. Nervous system manifestations include Lyme neuroborreliosis, most commonly presented as cranial neuropathy with facial nerve palsy, possibly with bilateral involvement (bilateral Bell's palsy), within a few weeks of infection. In adults, the disease typically presents as painful meningoradiculoneuritis and facial palsy. In contrast, children most frequently develop headache due to meningitis, and facial palsy. In children there are shorter lasting symptoms and better outcomes. Cardiac manifestations in LD appear to be uncommon, and Lyme carditis usually presents within two months of infection as myocarditis with acute intermittent atrioventricular heart block. In Europe, more severe skin manifestations (e.g. acrodermatitis chronica atrophicans (ACA), borrelial lymphocytoma) can result from disseminated infection as late complications [Stanek, et al. 2012, supra].

Outer surface protein A (OspA) is one of the dominant proteins expressed by Borrelia burgdorferi s.l. when present in an unfed tick. During tick feeding the incoming blood signals the down regulation of OspA expression, allowing the spirochetes to penetrate the gut epithelium, migrate to the salivary glands and further into the blood of the host [Schwan T G and Piesman J. Temporal changes in outer surface proteins A and C of the Lyme disease-associated spirochete, Borrelia burgdorferi, during the chain of infection in ticks and mice. J Clin Microbiol 2000,38:382-8]. OspA based LB vaccines induce antibodies that act on OspA expressing spirochetes within the tick gut, blocking transmission of the spirochete to the salivary glands and subsequently to the vertebrate host [de Silva A M and Telford S R 3rd, Brunet L R, Barthold S W, Fikrig E., J Exp Med. 1996, 183 (1): 271-5].

Two vaccines containing OspA from B. burgdorferi s.s. (ST1) have previously shown to be efficacious to prevent LB in humans: LYMErix (SmithKline Beecham) [Steere A C, et al., Lyme Disease Vaccine Study Group, Vaccination against Lyme disease with recombinant Borrelia burgdorferi outer-surface lipoprotein A with adjuvant, N Engl J Med. 1998; 339 (4): 209-15] and ImuLyme (Pasteur Mérieux Connaught) [Sigal L H, et al., A vaccine consisting of recombinant Borrelia burgdorferi outer-surface protein A to prevent Lyme disease, N Engl J Med. 1998; 339 (4): 216-22] LYMErix was licensed and available for costumers from 1998-2002, when it was voluntarily withdrawn from the market. After licensure of LYMErix, a possible relationship between the Lyme vaccine and joint reactions was hypothesized because of partial homology of OspA ST1 in the vaccine with hLFA-1 (human leukocyte function-associated antigen-1) that was claimed to induce antibiotic-refractory Lyme arthritis in a subset of naturally infected patients [Gross D M, et al., Identification of LFA-1 as a candidate autoantigen in treatment-resistant Lyme arthritis, Science. 1998 Jul. 31; 281 (5377): 703-6]. The hypothesis however could not be proven. On the contrary, a retrospective study of joint complaints reported to the Vaccine Adverse Event Reporting System (VAERS) after distribution of over 1,400,000 doses during the first 19 months after licensure of the vaccine showed no unusual number of such complaints [Lathrop S L, et al., Adverse event reports following vaccination for Lyme disease: December 1998-July 2000, Vaccine 20 (2002) 1603-1608]. In the Phase III study of the vaccine, the incidence of transient arthralgia was non-significantly increased in vaccinees, but the incidence of arthritis was not increased as compared to the placebo group [Steere, et al., 1998, supra].

More recently, Baxter conducted a Phase I/II study of a similar multivalent OspA based vaccine candidate, which has shown to be safe, well tolerated and highly immunogenic [Wressnigg N, et al.; Safety and immunogenicity of a novel multivalent OspA vaccine against Lyme borreliosis in healthy adults: a double-blind, randomised, dose-escalation phase 1/2 trial, Lancet Infect Dis. 2013 August; 13 (8): 680-9; Wressnigg N, et al., A Novel multivalent OspA vaccine against Lyme borreliosis is safe and immunogenic in an adult population previously infected with Borrelia burgdorferi sensu lato, Clin Vaccine Immunol. 2014 November; 21 (11): 1490-9]. However, this vaccine is currently not being developed further. Currently, there is no LB vaccine on the market and no other LB vaccine candidate is in clinical development.

The multivalent Lyme borreliosis vaccine of the current invention comprises the immunodominant C-terminal part of the six most common OspA serotypes present in Europe (ST1 to ST6) and the US (ST1) and is designed to induce anti-OspA antibodies to neutralize the Borrelia within the tick gut, blocking its transmission to the host. The multimeric Borrelia vaccine has been shown to be highly protective against four Borrelia genospecies (B. burgdorferi s.s., B. afzelii, B. bavariensis and B. garinii), including five clinically relevant OspA serotypes (ST1, ST2, ST4 to ST6) in mouse models using either infected ticks or in vitro grown spirochetes for challenge.

OspA based vaccines have shown to be efficacious in humans in the past. Valneva's multivalent Borrelia vaccine could prevent infection with the most common B. burgdorferi s.l. and OspA serotypes present in Europe and the US.

OspA-based vaccines require high and sustained antibody titers because of their mechanism of action (outside of the body).

### BRIEF DESCRIPTION OF THE DRAWINGS

FIG. 1. Serum bactericidal assay used as an in vitro functional assay (see Materials and Methods).

FIG. 2. Study design (First-in-human phase 1 study), including a booster at 13 months in selected subjects from the high-dose group (booster extension).

FIGS. 3A-3B. Phase 1 data: Immunogenicity data at day 84, Per-Protocol (PP) population (i.e. subjects that received all three immunizations at correct dose and correct timing according to protocol), for each serotype FIG. 3A) IgG geometric mean titer (GMT) and FIG. 3B) Seroconversion Rate, defined as percentage of subjects reaching a ≥4-fold increase in IgG titer from baseline.

FIGS. 4A-4D. Phase 1 data: IgG GMT values, Booster P P population (i.e., subjects enrolled in booster extension phase and received correct booster dose at correct timing according to protocol), for each serotype, at FIG. 4A) d84, FIG. 4B) M13, FIG. 4C) M14 and FIG. 4D) M19.

FIGS. 5A-5F. Phase 1 data: Serotype-specific IgG GMT values, Booster PP population, over time FIG. 5A) ST1, FIG. 5B) ST2, FIG. 5C) ST3, FIG. 5D) ST4, FIG. 5E) ST5, FIG. 5F) ST6.

FIGS. 6A-6C. Phase 1 data: Comparison of ELISA titers with SBA titers. FIG. 6A) Serotype 1 (ST1)—samples with ELISA titers lower than 100 U/mL rarely tested positive in the SBA. In total, 116 valid SBA data points were collected. The ST1 SBA lower limit of quantification (LLOQ) was set to 20 and negative samples were given a value of 10. FIG. 6B) ST2-samples with ELISA titers lower than 200 U/mL rarely tested positive in the SBA. In total, 48 valid SBA data points were collected. The ST2 SBA LLOQ was set to 20 and negative samples were given a value of 10. FIG. 6C) ST3-samples with ELISA titers lower than 100 U/mL rarely tested positive in the SBA. In total, 122 valid SBA data points were collected. The ST3 SBA LLOQ was set to 160 and negative samples were given a value of 80.

FIG. 7. First Phase 2 study: Study design (of first phase 2 trial) for the multivalent Borrelia vaccine of the invention, investigating 3 adjuvanted Vaccine A doses (90 μg, 135 μg and 180 μg) in a “Run-in phase” and 2 selected dose groups (135 μg, 180 μg) in a “Main Study phase”.

FIG. 8. Second Phase 2 study: Study design of second phase 2 trial for the multivalent Borrelia vaccine of the invention investigating an alternative immunization schedule.

FIGS. 9A-9F. Correlation ELISA-SBA titer, Day 208, pooled multimeric Borrelia vaccine treatment groups, PP population w/o baseline SBA seropositive subjects. (FIG. 9A) Spearman correlation coefficient: 0.7083, p-Value: <. 0001; (FIG. 9B) Spearman correlation coefficient: 0.8217, p-Value: <. 0001; (FIG. 9C) Spearman correlation coefficient: 0.9131, p-Value: <. 0001; (FIG. 9D) Spearman correlation coefficient: 0.6487, p-Value: <. 0001; (FIG. 9E) Spearman correlation coefficient: 0.6554, p-Value: <. 0001; (FIG. 9F) Spearman correlation coefficient: 0.7818, p-Value: <. 0001.

FIG. 10: Seroconversion Rates for OspA-specific IgG, per Serotype, D85, PP Population. Brackets Denote Significant Differences Between Groups. No significant differences observed between 135 μg and 180 μg treatment groups.

FIG. 11: Seroconversion Rates for OspA-specific IgG, per Serotype, D208, PP Population. Brackets Denote Significant Differences Between Groups. No significant differences observed between 135 μg and 180 μg treatment groups.

FIG. 12: Reverse Cumulative Distribution Curve for ELISA-ST1. Percentage of Subjects vs. OspA-specific IgG per time. Top panel: 135 μg treatment group. Bottom panel: 180 μg treatment group.

FIG. 13: Reverse Cumulative Distribution Curve for ELISA-ST2 Percentage of Subjects vs. OspA-specific IgG per time. Top panel: 135 μg treatment group. Bottom panel: 180 μg treatment group.

FIG. 14: Reverse Cumulative Distribution Curve for ELISA-ST3 Percentage of Subjects vs. OspA-specific IgG per time. Top panel: 135 μg treatment group. Bottom panel: 180 μg treatment group.

FIG. 15: Reverse Cumulative Distribution Curve for ELISA-ST4 Percentage of Subjects vs. OspA-specific IgG per time. Top panel: 135 μg treatment group. Bottom panel: 180 μg treatment group.

FIG. 16: Reverse Cumulative Distribution Curve for ELISA-ST5 Percentage of Subjects vs. OspA-specific IgG per time. Top panel: 135 μg treatment group. Bottom panel: 180 μg treatment group.

FIG. 17: Reverse Cumulative Distribution Curve for ELISA-ST6 Percentage of Subjects vs. OspA-specific IgG per time. Top panel: 135 μg treatment group. Bottom panel: 180 μg treatment group.

FIGS. 18A-18B: ELISA OspA-Specific IgG Antibodies (GMT) per Serotype over Time, PP Population. FIG. 18A) 135 μg group; FIG. 18B) 180 μg group.

FIG. 19: Subject enrollment for Phase 2 clinical trial for the multivalent Borrelia vaccine investigating a three-dose or two-dose primary immunization schedule that is performed in an age-descending, staggered manner for three age cohorts.

FIG. 20: Study design of Phase 2 clinical trial for the multivalent Borrelia vaccine investigating three-dose or two-dose primary immunization schedule of the vaccine, and booster dose, in a study population aged 5 to 65 years.

### DETAILED DESCRIPTION OF THE INVENTION

Therefore, in a first aspect, the present invention provides a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1) for use as a vaccine, wherein the vaccine is to be administered i) to a human adult at least three times at a total protein content of said 3 fusion proteins in the range of from 60 to 200 μg per dose or more preferably from 120 to 200 μg per dose or ii) to a human child at least three times at a total protein content of said 3 fusion proteins in the range of from 60 to 200 μg per dose or from 120 to 200 μg per dose or from 60 to 100 μg per dose. Alternatively, the present invention provides a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1) for use as a vaccine, wherein the vaccine is to be administered i) to a human adult at least two times at a total protein content of said 3 fusion proteins in the range of from 60 to 200 μg per dose or more preferably 120 to 200 μg per dose or ii) to a human child at least two times at a total protein content of said 3 fusion proteins in the range of from 60 to 200 μg per dose or 120 to 200 μg per dose or from 60 to 100 μg per dose. Said composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1) is herein also referred to as “Vaccine A” or the multimeric Borrelia vaccine (“MBV”), interchangeably.

The above fusion proteins (sometimes also referred to as heterodimers) are derived from borrelial outer surface protein A (OspA). OspA is expressed by Borrelia only when it is in the gut of the tick vector. Thus, OspA antibodies produced by vaccination do not fight infection in the body, but enter the gut of the tick when it takes a blood meal. There, the antibodies likely neutralize the spirochetes and block the migration of bacteria from the midgut to the salivary glands of the tick, the route through which Borrelia enters the vertebrate host. Thus, OspA-specific antibodies prevent the transmission of Borrelia from the tick vector to the human host.

The above proteins are lipidated mutant OspA fragment fusion proteins comprising a hybrid C-terminal OspA fragment, wherein the hybrid fragment consists of a C-terminal domain of OspA of Borrelia that is comprised of a fragment derived from OspA of a Borrelia strain different than B. garinii, strain PBr, and a second fragment of OspA from B. garinii, strain PBr, and differs from the corresponding wild-type sequence at least by the introduction of at least one disulfide bond. The disulfide bonds are disulfide bonds Type 1, wherein cysteine residues are inserted at position 183+/−3 and 270+/−3 (for further details see WO 2015/104396 A1). S3hyb indicates a fusion of amino acids 125-176 of B. valaisiana and amino acids 177-274 of B. garinii, strain PBr. Lip means lipidation and indicates the N-terminal addition of glycerol and fatty acid residues. The “LN1” peptide linker is a fusion of two separate loop regions of the N-terminal half of OspA from B. burgdorferi s.s., strain B31 (aa 65-74 and aa 42-53, with an amino acid exchange at position 53 of D53S) which has the following sequence: GTSDKNNGSGSKEKNKDGKYS (SEQ ID NO: 7).

Particularly, Lip-S1D1-S2D1 is a fusion protein of OspA serotype 1 and OspA serotype 2 with disulfide bond type 1 and comprises an N-terminal CSS for addition of lipids, a LN1 linker sequence and an N-terminal lipidation. Amino acids 164-174 of OspA serotype 1 are replaced by non-hLFA-1-like sequence NFTLEGKVAND (SEQ ID NO: 8). The sequence is shown as following SEQ ID NO: 1:

Lip-S1D1-S2D1-aa
SEQ ID NO: 1
LipCSSFNEKGEVSEKIITRADGTRLEYTGIKSDGSGKAKEVLKNFTLEGKVANDKTTLVVK
CGTVTLSKNISKSGEVSVELNDTDSSAATKKTAAWNSGTSTLTITVNSKKTKDLVFTKENTI
TVQQYDSNGTKLEGSAVEITKLDEICNALKGTSDKNNGSGSKEKNKDGKYSFNEKGELSAKT
MTRENGTKLEYTEMKSDGTGKAKEVLKNFTLEGKVANDKVTLEVKCGTVTLSKEIAKSGEVT
VALNDTNTTQATKKTGAWDSKTSTLTISVNSKKTTQLVFTKQDTITVQKYDSAGTNLEGTAV
EIKTLDELCNALK

Lip-S4D1-S3hybD1 is a fusion protein of OspA serotype 4 and OspA serotype 3, comprising amino acids 125-176 of B. valaisiana, strain VS116 and amino acids 177-274 of B. garinii, strain PBr, serotype 3, with disulfide bond type 1 and comprises an N-terminal CSS for addition of lipids, an LN1 linker sequence and N-terminal lipidation. The sequence is shown as following SEQ ID NO: 2:

Lip-S4D1-S3hybD1-aa
SEQ ID NO: 2
LipCSSFNAKGELSEKTILRANGTRLEYTEIKSDGTGKAKEVLKDFALEGTLAADKTTLKVT
CGTVVLSKHIPNSGEITVELNDSNSTQATKKTGKWDSNTSTLTISVNSKKTKNIVFTKEDTI
TVQKYDSAGTNLEGNAVEIKTLDELCNALKGTSDKNNGSGSKEKNKDGKYSFNEKGEVSEKI
LTRSNGTTLEYSQMTDAENATKAVETLKNGIKLPGNLVGGKTKLTVTCGTVTLSKNISKSGE
ITVALNDTETTPADKKTGEWKSDTSTLTISKNSQKTKQLVFTKENTITVQNYNRAGNALEGS
PAEIKDLAELCAALK

Lip-S5D1-S6D1 is a fusion protein of OspA serotypes 6 both with disulfide bond type 1, an N-terminal CSS for addition of lipids, LN1 linker sequence and an N-terminal lipidation. The sequence is shown as following SEQ ID NO: 3:

Lip-S5D1-S6D1-aa
SEQ ID NO: 3
LipCSSFNEKGEISEKTIVRANGTRLEYTDIKSDKTGKAKEVLKDFTLEGTLAADGKTTLKV
TCGTVTLSKNISKSGEITVALDDTDSSGNKKSGTWDSGTSTLTISKNRTKTKQLVFTKEDTI
TVQNYDSAGTNLEGKAVEITTLKELCNALKGTSDKNNGSGSKEKNKDGKYSFNGKGETSEKT
IVRANGTRLEYTDIKSDGSGKAKEVLKDFTLEGTLAADGKTTLKVTCGTVVLSKNILKSGEI
TAALDDSDTTRATKKTGKWDSKTSTLTISVNSQKTKNLVFTKEDTITVQRYDSAGTNLEGKA
VEITTLKELCNALK

The nucleic acid sequences coding for the above fusion proteins are as follows:

Lip-S1D1-S2D1-nt
SEQ ID NO: 4
ATGAAAGCTACTAAACTGGTACTGGGCGCGGTAATCCTGGGTTCTACTCTGCTGGCAGGTTG
CTCAAGCTTCAACGAAAAGGGCGAAGTCAGCGAAAAAATCATTACCCGCGCAGACGGCACCC
GCCTGGAATACACCGGCATCAAATCGGACGGCAGCGGCAAAGCGAAAGAAGTTCTGAAAAAC
TTTACCCTGGAAGGCAAAGTCGCAAATGATAAAACCACCCTGGTGGTGAAATGCGGCACCGT
TACGCTGAGCAAAAACATTAGTAAATCCGGTGAAGTCTCTGTGGAACTGAATGATACCGACA
GCTCTGCGGCCACCAAGAAAACCGCAGCTTGGAACTCAGGCACCTCGACGCTGACCATTACG
GTTAATAGCAAGAAAACCAAAGATCTGGTCTTCACGAAAGAAAACACCATCACGGTGCAGCA
ATATGACAGCAATGGTACCAAACTGGAAGGCTCCGCTGTGGAAATCACGAAACTGGATGAAA
TCTGTAATGCTCTGAAAGGTACTAGTGACAAAAACAATGGCTCTGGTAGCAAAGAGAAAAAC
AAAGATGGCAAGTACTCATTCAACGAAAAAGGCGAACTGTCGGCGAAAACGATGACGCGTGA
AAACGGCACCAAACTGGAATATACGGAAATGAAAAGCGATGGCACCGGTAAAGCGAAAGAAG
TTCTGAAAAACTTTACCCTGGAAGGCAAAGTCGCCAATGACAAAGTCACCCTGGAAGTGAAA
TGCGGCACCGTTACGCTGTCAAAAGAAATTGCAAAATCGGGTGAAGTGACCGTTGCTCTGAA
CGATACGAATACCACGCAAGCGACCAAGAAAACCGGCGCCTGGGACAGCAAAACCTCTACGC
TGACCATTAGTGTTAATAGCAAGAAAACCACGCAGCTGGTCTTCACCAAACAAGATACGATC
ACCGTGCAGAAATACGACAGTGCGGGTACCAACCTGGAAGGCACGGCTGTTGAAATCAAAAC
CCTGGACGAACTGTGTAACGCCCTGAAA
Lip-S4D1-S3hybD1-nt
SEQ ID NO: 5
ATGAAAGCTACTAAACTGGTACTGGGCGCGGTAATCCTGGGTTCTACTCTGCTGGCAGGTTG
CTCAAGCTTCAATGCTAAGGGCGAACTGAGCGAAAAAACGATCCTGCGTGCGAATGGCACCC
GTCTGGAATACACCGAAATCAAATCCGATGGTACGGGCAAAGCAAAGGAAGTCCTGAAAGAT
TTTGCTCTGGAAGGTACCCTGGCGGCCGACAAAACCACGCTGAAGGTGACGTGCGGCACCGT
GGTTCTGAGCAAACATATTCCGAACTCTGGTGAAATCACCGTTGAACTGAACGATAGCAATT
CTACGCAGGCAACCAAAAAGACGGGCAAATGGGACAGTAATACCTCCACGCTGACCATTTCA
GTCAACTCGAAAAAGACCAAAAATATTGTGTTCACGAAGGAAGATACGATCACCGTTCAAAA
ATATGACTCCGCGGGCACCAACCTGGAAGGCAATGCCGTCGAAATCAAAACCCTGGATGAAC
TGTGTAACGCCCTGAAGGGTACTAGTGACAAAAACAATGGCTCTGGTAGCAAAGAGAAAAAC
AAAGATGGCAAGTACTCATTCAACGAAAAAGGCGAAGTGAGCGAAAAAATTCTGACCCGTAG
CAATGGCACCACCCTGGAATATAGCCAGATGACCGATGCAGAAAATGCAACCAAAGCAGTTG
AAACCCTGAAAAACGGTATTAAACTGCCTGGTAATCTGGTTGGTGGTAAAACCAAACTGACC
GTTACCTGTGGCACCGTTACCCTGAGCAAAAACATTAGCAAAAGCGGTGAAATTACCGTGGC
ACTGAATGATACCGAAACCACACCGGCAGACAAAAAAACCGGTGAATGGAAAAGCGATACCA
GCACCCTGACCATTAGTAAAAATAGCCAGAAAACAAAACAGCTGGTGTTTACCAAAGAAAAC
ACCATTACCGTGCAGAATTATAACCGTGCAGGTAATGCACTGGAAGGTAGTCCGGCAGAAAT
TAAAGATCTGGCAGAACTGTGTGCAGCCCTGAAATAA
Lip-S5D1-S6D1-nt
SEQ ID NO: 6
ATGAAAGCTACTAAACTGGTACTGGGCGCGGTAATCCTGGGTTCTACTCTGCTGGCAGGTTG
CTCAAGCTTCAACGAAAAGGGCGAAATCTCAGAAAAAACCATCGTCCGCGCTAACGGCACCC
GCCTGGAATACACCGACATCAAATCAGACAAGACCGGTAAAGCGAAGGAAGTTCTGAAAGAT
TTTACGCTGGAAGGTACCCTGGCAGCAGACGGTAAAACCACGCTGAAGGTGACCTGCGGTAC
CGTTACGCTGTCCAAAAACATTAGTAAGTCCGGCGAAATCACGGTCGCCCTGGATGACACCG
ATAGCTCTGGCAACAAAAAGAGCGGTACCTGGGATTCAGGCACCTCGACGCTGACCATTTCT
AAAAATCGTACGAAAACCAAGCAGCTGGTCTTCACGAAAGAAGATACGATCACCGTGCAAAA
CTATGACAGCGCAGGTACCAATCTGGAAGGCAAAGCTGTGGAAATTACCACGCTGAAAGAAC
TGTGTAATGCTCTGAAAGGTACTAGTGACAAAAACAATGGCTCTGGTAGCAAAGAGAAAAAC
AAAGATGGCAAGTACTCATTCAACGGCAAAGGTGAAACGAGCGAAAAGACCATCGTGCGTGC
GAACGGTACCCGCCTGGAATATACGGACATTAAATCGGACGGCAGCGGCAAAGCAAAGGAAG
TCCTGAAAGATTTTACGCTGGAAGGTACCCTGGCAGCAGACGGTAAAACCACGCTGAAGGTG
ACGTGCGGCACCGTGGTTCTGTCAAAAAACATTCTGAAGTCGGGTGAAATCACCGCAGCTCT
GGATGACAGCGATACCACGCGTGCTACGAAAAAGACCGGTAAATGGGATAGCAAGACCTCTA
CGCTGACCATTAGTGTCAACTCCCAGAAAACGAAGAATCTGGTGTTCACCAAAGAAGATACG
ATCACCGTTCAACGCTATGACAGTGCGGGCACCAACCTGGAAGGCAAAGCCGTTGAAATTAC
CACGCTGAAAGAACTGTGTAATGCTCTGAAA

Further information on the fusion proteins and their production is derivable from WO 2015/104396 A1, wherein Lip-S1D1-S2D1, Lip-S4D1-S3hybD1 and Lip-S5D1-S6D1 correspond to SEQ ID NOs: 29, 27 and 33, respectively.

As detailed above, the fusion proteins are lipidated proteins, wherein the lipid moieties, along with the glycerol group, is also referred to as “Lip”. According to the invention, Lip comprises one to three lipids such as C₁₄₋₂₀ alkyl and/or C₁₄₋₂₀ alkenyl attached to a glycerol and an amino group of the N-terminal cysteine of the polypeptide of the invention, or preferably wherein Lip is a moiety of formula (I) below,

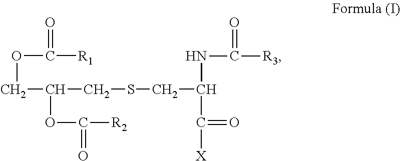

in which one of R₁, R₂ or R₃ is C₁₄-C₂₀ alkyl or alkenyl, and each of the others, independently is C₁₄-C₂₀ alkyl or C₁₄-C₂₀ alkenyl, and X is an amino acid sequence attached to the cysteine residue shown in Formula (I). More preferably, Lip plus the N-terminal cysteine of the polypeptide is N-palmitoyl-S-(2RS)-2,3-bis-(palmitoyloxy) propyl cysteine (referred to herein as “Pam3Cys”) and is connected via the carbonyl C of the cysteine to said amino acid sequence of the invention. In Formula (I) above R₁, R₂ and R₃ would be palmitoyl moieties and X is an amino acid sequence attached to the cysteine residue.

The fusion proteins are encompassed in a composition. The composition is pharmaceutically acceptable, which allows for administration to a human. It may optionally contain any pharmaceutically acceptable carrier or excipient, such as buffer substances, stabilizers or further active ingredients, especially ingredients known in connection with pharmaceutical compositions and/or vaccine production. The composition may comprise sodium phosphate, sodium chloride, L-methionine, sucrose and Polysorbate-20 (Tween-20) at a pH of 6.7+/−0.2.

Preferably, the pharmaceutical composition also comprises aluminium hydroxide, preferably at a concentration of 0.15%. Also the composition may comprise between 5 mM and 50 mM sodium phosphate, between 100 and 200 mM sodium chloride, between 5 mM and 25 mM L-methionine, between 2.5% and 10% Sucrose, between 0.01% and 0.1% Tween 20 and between 0.1% and 0.2% (w/v) aluminium hydroxide. More preferably, the formulation comprises 10 mM sodium phosphate, 150 mM sodium chloride, 10 mM L-methionine, 5% Sucrose, 0.05% Tween 20 and 0.15% (w/v) aluminium hydroxide at pH 6.7±0.2. In a preferred embodiment, the excipient is L-methionine.

In some embodiments, the composition comprising the fusion proteins is administered to a subject in a volume of about 0.25 milliliter (ml) to about 1.0 ml or greater, such as 0.25 ml, 0.3 ml, 0.4 ml, 0.5 ml, 0.6 ml, 0.7 ml, 0.8 ml, 0.9 ml, or 1.0 ml. In one embodiment, the volume administered to a subject is in the range of 0.25 ml to 1.0 ml, or 0.25 ml to 0.5 ml, or 0.5 ml to 1.0 ml. In one embodiment, the volume administered to a subject is about 0.25 ml, about 0.5 ml or about 1.0 ml. In one embodiment, the volume administered to a subject is 0.25 ml, 0.5 ml, or 1.0 ml. In one embodiment, the volume administered to a subject is 0.25 ml. In one embodiment, the volume administered to a subject is 0.5 ml. In one embodiment, the volume administered to a subject is 1.0 ml. In a preferred embodiment, the volume administered to a subject who is 5 years or older (such as 5-17 years old, 5-11 years old, 12-17 years old, 18 years or older, 18-65 years old, or 50 years or older) is 0.5 ml or 1.0 ml. In a preferred embodiment, the volume administered to a subject who is 5 years or older (such as 5-17 years old, 5-11 years old, 12-17 years old, 18 years or older, 18-65 years old, or 50 years or older) is 1.0 ml. In a further preferred embodiment, the volume administered to a subject who is 5 years or older (such as 5-17 years old, 5-11 years old, 12-17 years old, 18 years or older, 18-65 years old, or 50 years or older) is 0.5 ml. In a further preferred embodiment, the volume administered to a subject who is 0-4 years old (such as 1~4 years old, or 2-4 years old) is 0.25 ml.

According to the invention, the composition is used as a vaccine, particularly against an infection caused by Borrelia species, more preferably pathogenic Borrelia species as disclosed herein more preferably comprising B. burgdorferi s.s., B. afzelii, B. bavariensis and B. garinii, and/or other pathogens against which the antigens have been included in the vaccine. Preferably, the Borrelia species is selected from B. burgdorferi s.s., B. garinii, B. afzelii, B. andersoni, B. bavariensis, B. bissettii, B. valaisiana, B. lusitaniae, B. spielmanii, B. japonica, B. tanukii, B. turdi or B. sinica infection, preferably a B. burgdorferi s.s., B. afzelii and B. garinii.

It has been found that a specific administration schedule is advantageous, particularly in obtaining a sustained protective effect against all Borrelia OspA serotypes. This relates to the minimal number of administrations as well as the dose range. In accordance with that the vaccine is to be administered to a human adult at least three times at a total protein content of said 3 fusion proteins in the range of from 60 to 200 μg per dose or more preferably from 120 to 200 μg per dose or to a human child at least three times at a total protein content of said 3 fusion proteins in the range of from 60 to 200 μg per dose or from 120 to 200 μg per dose or from 60 to 100 μg per dose. In some embodiments, the vaccine is to be administered to a human adult at least two times at a total protein content of said 3 fusion proteins in the range of from 60 to 200 μg per dose or more preferably from 120 to 200 μg per dose or to a human child at least two times at a total protein content of said 3 fusion proteins in the range of from 60 to 200 μg per dose or from 120 to 200 μg per dose or from 60 to 100 μg per dose.

First data demonstrate that vaccines of the present invention such as Vaccine A at doses of 135 μg and 180 μg total protein content of the fusion proteins per dose were more effective than a dose of 90 μg (see Example 2). Accordingly, the total protein content of the 3 fusion protein is in the range of from 120 to 200 μg per dose for a human adult (adult dose). For children, half of the above doses may be applied, i.e. 60-100 μg (half dose), but the adult dose may also be appropriate i.e. 120 to 200 μg (adult dose). In context of administering half doses, a human child is typically a young child from birth until age 4 (0-4 years old), 1-4 years old, or 2-4 years old. Older children (age 5 to 17), including adolescents (age 12-17), receive the dose of an adult.

In view of the special mode of action of this vaccine which happens outside the human body, a need of constantly relatively high antibody titers throughout the tick seasons are of utmost importance. In addition, no natural boosting effect for present OspA-specific antibodies upon natural infection can be expected because OspA is only present on the surface of Borrelia as long as Borrelia are in the midgut of the tick and not after antibodies have possibly entered the human host. Therefore, high antibody titers are needed and the composition is to be administered at least three times, or alternatively two times. In accordance with this, the composition may be administered two times, three times, four times, five times or even more often.

The first primary immunization is referred herein to as the Month 0-2-6 immunization series.

The further administrations are booster doses for re-exposure to the vaccine and are applied typically every 12 months after the 3rd immunization of the primary immunization (i.e. first booster at month 18). Alternatively, the first primary immunization is referred herein to as the Month 0-6 immunization series. The further administrations are booster doses for re-exposure to the vaccine and are applied typically every 12 months after the 2nd immunization of the primary immunization (i.e. first booster at month 18). Booster doses increase immunity back to protective levels, after titers have declined through time (see Example 1). It has been shown that boosters are needed in case of the composition according to the present invention in order to sustain high OspA antibody levels which are needed to confer protection.

While booster doses typically are applied every 12 months, booster doses can be administered within several months of the 12 month timing, such as between 9-15 months after the final dose of the primary immunization (e.g., at 9 months, 10 months, 11 months, 12 months, 13 months, 14 months, or 15 months), or subsequent booster doses (e.g., at 9 months, 10 months, 11 months, 12 months, 13 months, 14 months, or 15 months after the first or subsequent booster doses).

The composition according to the present invention may be administered to the human as an injectable composition, for example as a sterile aqueous dispersion, preferably isotonic. The composition may be administered via a systemic or mucosal route. These administrations may include injection via the intramuscular, intraperitoneal, intradermal or subcutaneous routes; or via mucosal administration to the oral/alimentary, respiratory or genitourinary tracts. Although the vaccine of the invention may be administered as a combination of the 3 fusion proteins, components thereof (i.e. the individual fusion proteins) may also be administered separately at the same time or sequentially or just as a one component vaccine (i.e. just Lip-S1D1-S2D1).

In a second aspect, the present invention provides a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1) for use in a method for eliciting an immune response in a human against Lyme borreliosis, the method comprising administering to a human the composition at least three times at a total protein content of said 3 fusion proteins is in the range of from 120 to 200 μg per dose for a human adult or child or in case of a human child (in particular a young child 0 to 4 years old, 1-4 years old, or 2-4 years old) only half a dose such as e.g. at least three times at a total protein content of said 3 fusion proteins in the range of from 60 to 100 μg per dose. Alternatively, the present invention provides a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1) for use in a method for eliciting an immune response in a human against Lyme borreliosis, the method comprising administering to a human the composition at least two times at a total protein content of said 3 fusion proteins is in the range of from 120 to 200 μg per dose for a human adult or child or in case of a human child (in particular a young child 0 to 4 years old, 1-4 years old, or 2-4 years old) only half a dose such as e.g. at least two times at a total protein content of said 3 fusion proteins in the range of from 60 to 100 μg per dose.

The above definitions and comments made with respect to the first aspect of the invention apply also to the second aspect of the invention.

In the second aspect, the composition is used in a method for eliciting an immune response in a human against Lyme disease caused by Borrelia species, more preferably pathogenic Borrelia species as disclosed herein more preferably comprising B. burgdorferi s.s., B. afzelii, B. bavariensis and B. garinii, and/or other pathogens against which the antigens have been included in the composition. Preferably, the composition is for use in a method of eliciting an immune response against B. burgdorferi s.s., B. garinii, B. afzelii, B. andersoni, B. bavariensis, B. bissettii, B. valaisiana, B. lusitaniae, B. spielmanii, B. japonica, B. tanukii, B. turdi or B. sinica infection, preferably a B. burgdorferi s.s., B. afzelii and/or B. garinii.

Lyme borreliosis, or Lyme disease, is the most commonly reported tick-borne disease in Europe and North America. The disease is caused by infection with the arthropod-borne gram-negative-like spirochete, Borrelia burgdorferi sensu lato (B. burgdorferi s.l.), and can involve multiple organs or tissues, resulting in skin, cardiac, musculoskeletal and neurological disorders. In most countries, Lyme borreliosis is not a notifiable disease; therefore, exact data regarding annual incident rates are not available. In the United States, the causative agent is B. burgdorferi sensu stricto (B. burgdorferi s.s.) and Lyme borreliosis is localized to north-eastern, mid-Atlantic and upper north-central states. In 2010, a total of about 30,000 cases of Lyme borreliosis were reported to the US to the Centers for Disease Control and Prevention (CDC). An updated report by the CDC in 2013, which takes into account diagnostic data from other sources, estimates that the actual number of new cases per year in the United States is closer to 300,000 (cdc.gov/media/releases/2013/p0819-lyme-disease.html). In Europe, B. afzelii and B. garinii are the main causative agents of Lyme borreliosis, as well as B. burgdorferi s.s. and B. bavariensis, which contribute to a lesser extent depending on the geographic location. The prevalence of Lyme borreliosis varies considerably in different European countries with an overall increased prevalence from west to east. In much of Europe, the number of reported cases of Lyme borreliosis has increased since the early 1990s (e.g., the Czech Republic, Estonia, Lithuania; see Lyme borreliosis in Europe, WHO report of 2006), and the geographic distribution of cases has also expanded.

As detailed above, high antibody titers after primary immunization and after booster doses are needed to sustain high OspA antibody levels. Particularly, a broad primary immunization schedule (e.g. Month 0-2-6) has been found suitable in the context of the present invention (see Example 3). Alternatively, a two dose primary immunization schedule (e.g. Month 0-6) may be used.

Therefore, the above compositions for use according to the present invention are preferably characterized in that the second administration is in the period of at least 4 to 6 weeks to at most 3 months after the first administration and/or the third administration is in the period of at least 5 months to at most 7 months after the first administration. In accordance with this, the first administration occurs at time point zero (day 1), the second administration in the period of at least 6 weeks to at most 3 months thereafter and the third administration in the period of at least 5 months to at most 7 months after the first administration. Further booster may be every year, e.g. 12 months, 24 months, 36 months etc. after the 3rd vaccine administration (i.e. after the month 6 immunization). Alternatively, the second administration is in the period of at least 5 months to at most 7 months after the first administration. In accordance with this, the first administration occurs at time point zero (day 1), the second administration in the period of at least 5 months to at most 7 months after the first administration. Further booster may be every year, e.g. 12 months, 24 months, 36 months etc. after the second vaccine administration (i.e. after the month 6 immunization). Accordingly, compositions for use according to the present invention are preferably characterized in that further administrations are every year after the third administration (or alternatively, the second administration in a Month 0-6 two dose primary immunization schedule), particularly after 1 year, after 2 years and after 3 years, etc. Said booster doses may in general be administered prior to the tick season, i.e. around February to June in the Northern hemisphere and around September to December in the Southern hemisphere.

More preferably, the above compositions for use according to the present invention are characterized in that the second administration is in the period of at least 40 days to at most 80 days, particularly in the period of at least 50 days to at most 70 days, more particularly in the period of at least 50 days to at most 60 days, especially 56 days after the first administration.

Also more preferably, the above compositions for use according to the present invention are characterized in that the third administration is in the period of at least 120 days to at most 240 days, in the period of at least 175 days to at most 185 days, especially 180 days after the first administration. Alternatively, the above compositions for use according to the present invention are characterized in that the second administration is in the period of at least 120 days to at most 240 days, in the period of at least 175 days to at most 185 days, especially 180 days after the first administration.

In some cases, one or more further administrations may be needed or desirable. In accordance with this a fourth administration of the composition (or alternatively, a third administration in a Month 0-6 two dose primary immunization schedule) may be in the period of at least 15 months to at most 21 months, particularly in the period of at least 17 months to at most 19 months, especially 18 months, after the first administration.

Preferably, the above compositions for use according to the present invention are characterized in that for a human adult (age 18 or older, including age 18-65), the total protein content of said 3 fusion proteins is in the range of from 135 μg to 180 μg per dose, particularly 135 μg or 180 μg per dose. The doses of 135 μg and 180 μg total protein content of said 3 fusion proteins have been particularly suitable in effecting a sustained protective effect against all Borrelia OspA serotypes (see Examples 2 and 3). Accordingly, it can be concluded that the effect is present for the complete range of from 135 μg to 180 μg total protein content of said 3 heterodimers per dose. As detailed above, for a young child (birth to 4 years old) half of the doses may apply (but a full dose may also be suitable). Accordingly, for a human child (in particular, a young child of birth to 4 years old, e.g., 1-4 years old, or 2-4 years old), the total protein content of said 3 fusion proteins is in the range of from 67.5 μg to 90 μg per dose, particularly 67.5 μg or 90 μg per dose. For older children (age 5-17), including adolescents (age 12-17), a full dose of 135 μg to 180 μg total protein content of said 3 heterodimers per dose can be used.

Preferably, the above compositions for use according to the present invention are characterized in that the 3 fusion proteins comprise at least 60%, preferably at least 70%, more preferably 80% of all proteins in the composition. It is apparent to the person skilled in the art that the composition for use according to the present invention may also encompass further proteins different from the fusion proteins of SEQ ID NOs: 1, 2 and 3. However, the ratio of the fusion proteins should not be below the above limits, based on the weight. Other proteins may have a function, e.g. in stabilizing the composition or may be impurities.

In one embodiment, the pharmaceutical composition comprises 3 fusion proteins, preferably Lip-S1D1-S2D1 (SEQ ID NO: 1), Lip-S4D1-S3hybD1 (SEQ ID NO: 2) and Lip-S5D1-S6D1 (SEQ ID NO: 3) in a weight ratio of 1:2:1, 1:3:1, 1:1:2, 1:1:3, 1:2:2, 1:2:3, 1:3:2, 1:3:3, 2:1:1, 2:1:2, 2:1:3, 2:2:3, 2:2:1, 2:3:1, 2:3:2, 2:3:3, 3:1:1, 3:1:2, 3:1:3, 3:2:1, 3:2:2, 3:2:3, 3:3:1, or 3:3:2. Preferably, the above compositions for use according to the present invention are characterized in that the composition comprises the fusion proteins in a weight ratio of 1:1:1 (Lip-S1D1-S2D1: Lip-S4D1-S3hybD1: Lip-S5D1-S6D1).

Preferably, the above compositions for use according to the present invention are characterized in that the composition comprises an adjuvant, more preferably an aluminum adjuvant.

The choice of a suitable adjuvant to be mixed with bacterial proteins made using the processes of the invention is within the knowledge of the person skilled in the art. Suitable adjuvants include an aluminium salt such as aluminium hydroxide or aluminum phosphate, but may also be other metal salts such as those of calcium, magnesium, iron or zinc, or may be an insoluble suspension of acylated tyrosine, or acylated sugars, cationically or anionically derivatized saccharides, or polyphosphazenes. In a preferred embodiment, the composition is adjuvanted with an aluminium adjuvant, such as aluminium hydroxide. In a further preferred embodiment, the amount of copper associated in the aluminium adjuvant is less than 1.25 ppb in the vaccine composition.

Most preferably, the above compositions for use according to the present invention are characterized in that the total protein content of said 3 fusion protein is 135 μg per dose for a human subject, particularly an adult or a child, or may be only 67.5 μg per dose for a human child (in particular, a young child from birth to 4 years, 1-4 years old, or 2-4 years old), wherein the second administration is 56 days (2 months) after the first administration, wherein the third administration is 180 days (6 months) after the first administration, and wherein the optional fourth administration (1^{st} booster dose) is 18 months after the first administration.

Also most preferably, the above compositions for use according to the present invention are characterized in that the total protein content of said 3 fusion proteins is 180 μg per dose for a human subject, particularly an adult or a child, or may be 90 μg per dose for a human child (in particular, a young child from birth to 4 years, 1-4 years old, or 2-4 years old), wherein the second administration is 56 days (2 months) after the first administration, wherein the third administration is 180 days (6 months) after the first administration, and wherein the optional fourth administration (first booster dose) is 18 months after the first administration.

Alternatively, the above compositions for use according to the present invention are characterized in that the total protein content of said 3 fusion protein is 135 μg per dose for a human subject, particularly an adult or a child, or may be only 67.5 μg per dose for a human child (in particular, a young child from birth to 4 years, 1-4 years old, or 2-4 years old), wherein the second administration is 180 days (6 months) after the first administration, and wherein the optional third administration (1^{st} booster dose) is 18 months after the first administration.

Also alternatively, the above compositions for use according to the present invention are characterized in that the total protein content of said 3 fusion proteins is 180 μg per dose for a human subject, particularly an adult or a child, or may be 90 μg per dose for a human child (in particular, a young child from birth to 4 years, 1-4 years old, or 2-4 years old), wherein the second administration is 180 days (6 months) after the first administration, and wherein the optional third administration (first booster dose) is 18 months after the first administration.

Preferably, the above compositions for use according to the present invention are characterized in that the immune response elicited comprises an anti-OspA serotype 1, an anti-OspA serotype 2, an anti-OspA serotype 3, an anti-OspA serotype, anti-OspA serotype 5 and/or an anti-OspA serotype 6 antibody response with bactericidal activity . . .

Preferably, the above compositions for use according to the present invention are characterized in that the immune response elicited comprises antibodies with bactericidal activity against OspA serotypes 1, 2, 3, 4, 5 and 6 expressing spirochetes.

Preferably, the above compositions for use according to the present invention are characterized in that the immune response against OspA serotypes 1, 2, 3, 4, 5 and 6 is sustained for at least about 60 days, for at least about 180 days, for at least about 365 days or for at least about 540 days. Sustained immune response may be tested as described in Example 2.

In one embodiment the composition or vaccine of the invention further comprises at least one additional antigen (herein referred to generically as “combination vaccine”). In a preferred embodiment, the at least one additional antigen is derived from a Borrelia species causing Lyme borreliosis. In various aspects, the at least one additional antigen is derived from another pathogen, preferably a tick-borne pathogen. In a further aspect, the pathogen causes Rocky Mountain spotted fever, Human granulocytic ehrlichiosis (HGE), Sennetsu Fever, Human Monocytic Ehrlichiosis (HME), Anaplasmosis, Boutonneuse fever, Rickettsia parkeri Rickettsiosis, Southern Tick-Associated Rash Illness (STARI), Helvetica Spotted fever, 364D Rickettsiosis, African spotted fever, Relapsing fever, Tularemia, Colorado tick fever, Tick-borne encephalitis (TBE, also known as FSME), Crimean-Congo hemorrhagic fever, Q fever, Omsk hemorrhagic fever, Kyasanur forest disease, Powassan encephalitis, Heartland virus disease or Babesiosis. In a further aspect, the disease is Japanese encephalitis. In a further embodiment, the at least one additional antigen is derived from a vector-borne, preferably a tick-borne, pathogen selected from the group comprising Borrelia hermsii, Borrelia parkeri, Borrelia duttoni, Borrelia miyamotoi, Borrelia turicatae, Rickettsia rickettsii, Rickettsia australis, Rickettsia conori, Rickettsia helvetica, Francisella tularensis, Anaplasma phagocytophilum, Ehrlichia sennetsu, Ehrlichia chaffeensis, Neoehrlichia mikurensis, Coxiella burnetii and Borrelia lonestari, Tick-borne encephalitis virus (TBEV aka FSME virus), Colorado tick fever virus (CTFV), Crimean-Congo hemorrhagic fever virus (CCHFV), Omsk Hemorrhagic Fever virus (OHFV), Japanese encephalitis virus (JEV) and Babesia spp.

In one embodiment, the composition for use according to the present invention provides the use in an elderly subject with a similar immunogenic profile as in a younger subject, comprises wherein said human adult is an elderly subject who is older than 50 years. The invention surprisingly provides that older adults (age group 50-65 years) have shown similar immune responses compared to younger adults (age group 18-49 years), without statistical significant differences. Refer to the experimental part.

In one embodiment, the composition for use according to the present invention comprises less than 1.25 ppb copper or a sufficient amount of L-methionine. Aluminum is an adjuvant often used in vaccination. The aluminum adjuvants typically comprise impurities, particularly heavy metals, such as copper, nickel and iron. The presence of these, particularly of coppers, lowers the bioavailability of the OspA protein in the vaccine. Without being bound to the theory it is assumed that the OspA protein binds to the aluminum and the heavy metal, particularly the copper, prevents the release, thus decreasing the bioavailability of the OspA protein in the vaccine. Therefore, the composition and in particular the aluminum adjuvant comprises less than 1.25 ppb copper. The unit ppb (part per billion) is often used in the field of mass spectrometry to quantify impurities. In case of aqueous solutions, 1 ppb means that 1 ng of substance (impurity) is present in 1 g solution, which means that 1 ppb equals 1 μg/l (assuming that 1 liter of solution has a weight of 1 kg). Typically, copper is in form of an ion, particularly as Cu⁺ or Cu²⁺. In a preferred embodiment, the composition comprises less than 1.00 ppb, less than 0.75 ppb or less than less than 0.50 ppb copper based on the weight of the aqueous composition. It is known that L-methionine is capable of binding to copper. The amount of L-methionine required will evidently depend from the amount of copper in the composition. The person skilled in the art will be capable of selecting a suitable amount of L-methionine. L-methionine may be used to complex excess copper in the composition for use in the present invention. A typical suitable concentration of L-methionine in the composition is at least 10 mmol/l. Depending on the amount of copper in the composition, the concentration may be even higher such as at least 20 mmol/l, at least 30 mmol/l, at least 40 mmol/l or at least 50 mmol/l or lower such as at most 10 mmol/l, at most 5 mmol/l or at most 1 mmol/l. Alternatively, the concentration of L-methionine is determined based on the concentration of copper in the composition. Particularly, the concentration of L-methionine in mol/l is at least equivalent with the concentration of copper in the composition. Alternatively, the concentration of L-methionine in mol/l is at least twice, threefold, fourfold, fivefold, or even tenfold, the concentration of copper in the composition.

In one embodiment, composition for use further comprises a reactive compound, wherein the reactive compound is selected from the group consisting of a redox active compound, a radical building compound, a stabilizing compound and a combination of any thereof. Without being bound to theory, antigen degradation of protein vaccines in aqueous compositions comprising heavy metal ions present in an aluminium salt, such as aluminium hydroxide, might be explained with an underlying degradation pathway assuming free-radicals such as e.g. free-radicals of sulphite. Heavy metal-catalysed oxidation is a degradation pathway resulting in covalent modification of proteins. The modified physicochemical properties of the oxidized/modified protein or antigen may result in loss of biological activity. Redox active compounds are suitable to prevent this modification. Preferably, the reactive compound is selected from the group consisting of formaldehyde, ethanol, chloroform, trichloroethylene, acetone, triton-X-100, deoxycholate, diethylpyrocarbonate, sulphite, Na₂S205, beta-proprio-lacton, polysorbate such as Tween 20®, Tween 80®, O₂, phenol, pluronic type copolymers, and a combination of any thereof.

### Further Embodiments of the Present Invention are as Follows

1. A method for vaccinating a human, the method comprising administering a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1)
   to a human adult at least three times at a total protein content of said 3 fusion proteins in the range of from 120 to 200 μg per dose; or
   to a human child at least three times at a total protein content of said 3 fusion proteins in the range of from 120 to 200 μg per dose or from 60 to 100 μg per dose.
2. A method for eliciting an immune response in a human against Lyme disease, the method comprising administering a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1)
   to a human adult at least three times at a total protein content of said 3 fusion proteins in the range of from 120 to 200 μg per dose; or
   to a human child at least three times at a total protein content of said 3 fusion proteins in the range of from 120 to 200 μg per dose or from 60 to 100 μg per dose.
3. The method according to embodiment 1 or embodiment 2, wherein the second administration is in the period of at least 6 weeks to at most 3 months after the first administration and/or the third administration is in the period of at least 5 months to at most 7 months after the first administration.
4. The method according to any of embodiments 1 to 3, wherein the second administration is in the period of at least 50 days to at most 70 days, particularly in the period of at least 55 days to at most 60 days, especially 56 days after the first administration; and/or wherein the third administration is in the period of at least 170 days to at most 190 days, in the period of at least 175 days to at most 185 days, especially 180 day, after the first administration.
5. The method according to any of embodiments 1 to 4, wherein a fourth administration of the composition is in the period of at least 15 months to at most 21 months, particularly in the period of at least 17 months to at most 19 months, especially 18 months, after the first administration.
6. The method according to any of embodiments 1 to 5, wherein the total protein content of said 3 fusion proteins is in the range of from 135 μg to 180 μg per dose, particularly 135 μg or 180 μg per dose for a human adult or child, or wherein the total protein content of said 3 fusion proteins is in the range of from 67.5 μg to 90 μg per dose, particularly 67.5 μg or 90 μg per dose for a young child.
7. The method according to any of embodiments 1 to 6, wherein the 3 fusion proteins comprise at least 60%, preferably at least 70%, more preferably at least 80% of all proteins in the composition.
8. The method according to any of embodiments 1 to 7, wherein the composition comprises the fusion proteins in a weight ratio of 1:1:1 (Lip-S1D1-S2D1: Lip-S4D1-S3hybD1: Lip-S5D1-S6D1).
9. The method according to any of embodiments 1 to 8, wherein the composition comprises an adjuvant.
10. The method according to any of embodiments 1 to 9, wherein the composition comprises an aluminum adjuvant.
11. The method according to any of embodiments 1 to 10, wherein the total protein content of said 3 fusion proteins is 135 μg per dose for a human adult or child, wherein the second administration is 56 days after the first administration, wherein the third administration is 180 days after the first administration, and wherein the optional fourth administration is 18 months after the first administration and optionally thereafter every 12 months.
12. The method according to any of embodiments 1 to 10, wherein the total protein content of said 3 fusion proteins is 67.5 μg per dose for a young child, wherein the second administration is 56 days after the first administration, wherein the third administration is 180 days after the first administration, and wherein the optional fourth administration is 18 months after the first administration and optionally thereafter every 12 months.
13. The method according to any of embodiments 1 to 10, wherein the total protein content of said 3 fusion proteins is 180 μg per dose for a human adult or child, wherein the second administration is 56 days after the first administration, wherein the third administration is 180 days after the first administration, and wherein the optional fourth administration is 18 months after the first administration and optionally thereafter every 12 months.
14. The method according to any of embodiments 1 to 10, wherein the total protein content of said 3 fusion proteins is 90 μg per dose for a young child, wherein the second administration is 56 day after the first administration, wherein the third administration is 180 days after the first administration, and wherein the optional fourth administration is 18 months after the first administration and optionally thereafter every 12 months.
15. The method according to any of embodiments 1 to 10, wherein the total protein content of said 3 fusion proteins is 135 μg per dose for a human adult or child, wherein the second administration is 2 months after the first administration, wherein the third administration is 6 months after the first administration, and wherein the optional fourth administration is 18 months after the first administration and optionally thereafter every 12 months.
16. The method according to any of embodiments 1 to 10, wherein the total protein content of said 3 fusion proteins is 67.5 μg per dose for a young child, wherein the second administration is 2 months after the first administration, wherein the third administration is 6 months after the first administration, and wherein the optional fourth administration is 18 months after the first administration and optionally thereafter every 12 months.
17. The method according to any of embodiments 1 to 10, wherein the total protein content of said 3 fusion proteins is 180 μg per dose for a human adult or child, wherein the second administration is 2 months after the first administration, wherein the third administration is 6 months the first administration, and wherein the optional fourth administration is 18 months after the first administration and optionally thereafter every 12 months.
18. The method according to any of embodiments 1 to 10, wherein the total protein content of said 3 fusion proteins is 90 μg per dose for a young child, wherein the second administration is 2 months after the first administration, wherein the third administration is 6 months after the first administration, and wherein the optional fourth administration is 18 months after the first administration and optionally thereafter every 12 months.
19. The method according to any of embodiments 1 to 18, wherein further administrations are every year after the last primary immunization or at the beginning of each tick season, particularly after 1 year, after 2 years and after 3 years and so on.
20. The method according to any of embodiments 1 to 19, wherein the immune response elicited comprises an anti-OspA serotype 1, an anti-OspA serotype 2, an anti-OspA serotype 3, an anti-OspA serotype 4, anti-OspA serotype 5 and/or an anti-OspA serotype 6 antibody response with bactericidal activity.
21. The method according to any of embodiments 1 to 20, wherein the immune response elicited comprises antibodies with bactericidal activity against OspA serotypes 1, 2, 3, 4, 5 and 6 expressing spirochetes.
22. The method according to any of embodiments 1 to 21, wherein the immune response against OspA serotypes 1, 2, 3, 4, 5 and 6 is sustained for at least about 60 days, for at least about 180 days, for at least about 365 days or for at least about 540 days.
23. The method according to any of embodiments 1 to 22, wherein the composition comprises less than 1.25 ppb copper or a sufficient amount of L-methionine.
24. The method according to any of embodiments 1 to 23, wherein copper is in form of an ion, particularly as Cu⁺ or Cu²⁺.
25. The method according to any of embodiments 1 to 24, wherein L-methionine is present in a concentration of at least 10 mmol/l.
26. The method according to any of embodiments 23 to 25, wherein the concentration of L-methionine in mol/l is at least equivalent with the concentration of copper in the composition.
27. The method according to any of embodiments 1 to 26, wherein the composition further comprises a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton-X-100, deoxycholate, diethylpyrocarbonate, sulphite, Na₂S205, beta-proprio-lacton, polysorbate such as Tween 20®, Tween 80®, O₂, phenol, pluronic type copolymers, and a combination of any thereof.
28. The method according to any of embodiments 1 to 27, wherein said human adult is a subject who is 18 years or older, such as 18-65 years old.
29. The method according to any of embodiments 1 to 27, wherein said human adult is an elderly subject who is 50 years or older.
30. The method according to any of embodiments 1 to 27, wherein said human child is a subject who is 5-17 years old, such as 12-17 years old or 5-11 years old.
31. The method according to any of embodiments 1 to 27, wherein said human child is a subject who is 0-4 years old, such as 1~4 years old, or 2-4 years old.
32. The method according to any of embodiments 1 to 31, wherein said composition is administered to a human adult or child in a volume of 0.25 milliliter (ml) to 1.0 ml, such as 0.25 ml, 0.3 ml, 0.4 ml, 0.5 ml, 0.6 ml, 0.7 ml, 0.8 ml, 0.9 ml, or 1.0 ml.
33. The method according to any of embodiments 1 to 32, wherein said composition is administered to a human adult or child in a volume of 0.25 ml, 0.5 ml, or 1.0 ml.
34. A method for vaccinating a human, the method comprising administering a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1)
   to a human adult at least two times at a total protein content of said 3 fusion proteins in the range of from 120 to 200 μg per dose; or
   to a human child at least two times at a total protein content of said 3 fusion proteins in the range of from 120 to 200 μg per dose or from 60 to 100 μg per dose.
35. A method for eliciting an immune response in a human against Lyme disease, the method comprising administering a composition comprising the fusion protein of SEQ ID NO: 1 (Lip-S1D1-S2D1), the fusion protein of SEQ ID NO: 2 (Lip-S4D1-S3hybD1) and the fusion protein of SEQ ID NO: 3 (Lip-S5D1-S6D1)
   to a human adult at least two times at a total protein content of said 3 fusion proteins in the range of from 120 to 200 μg per dose; or
   to a human child at least two times at a total protein content of said 3 fusion proteins in the range of from 120 to 200 μg per dose or from 60 to 100 μg per dose.
36. The method according to embodiment 34 or embodiment 35, wherein the second administration is in the period of at least 5 months to at most 7 months after the first administration.
37. The method according to any of embodiments 34 to 36, wherein the second administration is in the period of at least 170 days to at most 190 days, in the period of at least 175 days to at most 185 days, especially 180 days, after the first administration.
38. The method according to any of embodiments 34 to 37, wherein a third administration of the composition is in the period of at least 15 months to at most 21 months, particularly in the period of at least 17 months to at most 19 months, especially 18 months, after the first administration.
39. The method according to any of embodiments 34 to 38, wherein the total protein content of said 3 fusion proteins is in the range of from 135 μg to 180 μg per dose, particularly 135 μg or 180 μg per dose, or wherein the total protein content of said 3 fusion proteins is in the range of from 67.5 μg to 90 μg per dose, particularly 67.5 μg or 90 μg per dose for a young child.
40. The method according to any of embodiments 34 to 39, wherein the 3 fusion proteins comprise at least 60%, preferably at least 70%, more preferably at least 80% of all proteins in the composition.
41. The method according to any of embodiments 34 to 40, wherein the composition comprises the fusion proteins in a weight ratio of 1:1:1 (Lip-S1D1-S2D1: Lip-S4D1-S3hybD1: Lip-S5D1-S6D1).
42. The method according to any of embodiments 34 to 41, wherein the composition comprises an adjuvant.
43. The method according to any of embodiments 34 to 42, wherein the composition comprises an aluminum adjuvant.
44. The method according to any of embodiments 34 to 43, wherein the total protein content of said 3 fusion proteins is 135 μg per dose for a human adult or child, wherein the second administration is 180 days after the first administration, and wherein the optional third administration is 18 months after the first administration and optionally thereafter every 12 months.
45. The method according to any of embodiments 34 to 43, wherein the total protein content of said 3 fusion proteins is 67.5 μg per dose for a young child, wherein the second administration is 180 days after the first administration, and wherein the optional third administration is 18 months after the first administration and optionally thereafter every 12 months.
46. The method according to any of embodiments 34 to 43, wherein the total protein content of said 3 fusion proteins is 180 μg per dose for a human adult or child, wherein the second administration is 180 days after the first administration, and wherein the optional third administration is 18 months after the first administration and optionally thereafter every 12 months.
47. The method according to any of embodiments 34 to 43, wherein the total protein content of said 3 fusion proteins is 90 μg per dose for a young child, wherein the second administration is 180 days after the first administration, and wherein the optional third administration is 18 months after the first administration and optionally thereafter every 12 months.
48. The method according to any of embodiments 34 to 43, wherein the total protein content of said 3 fusion proteins is 135 μg per dose for a human adult or child, wherein the second administration is 6 months after the first administration, and wherein the optional third administration is 18 months after the first administration and optionally thereafter every 12 months.
49. The method according to any of embodiments 34 to 43, wherein the total protein content of said 3 fusion proteins is 67.5 μg per dose for a young child, wherein the second administration is 6 months after the first administration, and wherein the optional third administration is 18 months after the first administration and optionally thereafter every 12 months.
50. The method according to any of embodiments 34 to 43, wherein the total protein content of said 3 fusion proteins is 180 μg per dose for a human adult or child, wherein the second administration is 6 months the first administration, and wherein the optional third administration is 18 months after the first administration and optionally thereafter every 12 months.
51. The method according to any of embodiments 34 to 43, wherein the total protein content of said 3 fusion proteins is 90 μg per dose for a young child, wherein the second administration is 6 months after the first administration, and wherein the optional third administration is 18 months after the first administration and optionally thereafter every 12 months.
52. The method according to any of embodiments 34 to 51, wherein further administrations are every year after the last primary immunization or at the beginning of each tick season, particularly after 1 year, after 2 years and after 3 years and so on.
53. The method according to any of embodiments 34 to 52, wherein the immune response elicited comprises an anti-OspA serotype 1, an anti-OspA serotype 2, an anti-OspA serotype 3, an anti-OspA serotype 4, anti-OspA serotype 5 and/or an anti-OspA serotype 6 antibody response with bactericidal activity.
54. The method according to any of embodiments 34 to 53, wherein the immune response elicited comprises antibodies with bactericidal activity against OspA serotypes 1, 2, 3, 4, 5 and 6 expressing spirochetes.
55. The method according to any of embodiments 34 to 54, wherein the immune response against OspA serotypes 1, 2, 3, 4, 5 and 6 is sustained for at least about 60 days, for at least about 180 days, for at least about 365 days or for at least about 540 days.
56. The method according to any of embodiments 34 to 55, wherein the composition comprises less than 1.25 ppb copper or a sufficient amount of L-methionine.
57. The method according to any of embodiments 34 to 56, wherein copper is in form of an ion, particularly as Cu⁺ or Cu²⁺.
58. The method according to any of embodiments 34 to 57, wherein L-methionine is present in a concentration of at least 10 mmol/l.
59. The method according to any of embodiments 56 to 58, wherein the concentration of L-methionine in mol/l is at least equivalent with the concentration of copper in the composition.
60. The method according to any of embodiments 34 to 59, wherein the composition further comprises a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, triton-X-100, deoxycholate, diethylpyrocarbonate, sulphite, Na₂S205, beta-proprio-lacton, polysorbate such as Tween 20®, Tween 80®, O₂, phenol, pluronic type copolymers, and a combination of any thereof.
61. The method according to any of embodiments 34 to 60, wherein said human adult is a subject who is 18 years or older, such as 18-65 years old.
62. The method according to any of embodiments 34 to 60, wherein said human adult is an elderly subject who is 50 years or older.
63. The method according to any of embodiments 34 to 60, wherein said human child is a subject who is 5-17 years old, such as 12-17 years old or 5-11 years old.
64. The method according to any of embodiments 34 to 60, wherein said human child is a subject who is 0-4 years old, such as 1~4 years old, or 2-4 years old.
65. The method according to any of embodiments 34 to 64, wherein said composition is administered to a human adult or child in a volume of 0.25 milliliter (ml) to 1.0 ml, such as 0.25 ml, 0.3 ml, 0.4 ml, 0.5 ml, 0.6 ml, 0.7 ml, 0.8 ml, 0.9 ml, or 1.0 ml.
66. The method according to any of embodiments 34 to 65, wherein said composition is administered to a human adult or child in a volume of 0.25 ml, 0.5 ml, or 1.0 ml.

The terms “comprising”, “comprise” and “comprises” herein are intended by the inventors to be optionally substitutable with the terms “consisting of”, “consist of” and “consists of”, respectively, in every instance. The term “comprises” means “includes”. Thus, unless the context requires otherwise, the word “comprises”, and variations such as “comprise” and “comprising” will be understood to imply the inclusion of a stated compound or composition (e.g., nucleic acid, polypeptide, antibody) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example”.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a”, “an”, and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, may be approximate.

The present invention is further illustrated by the following Figures, Tables and Examples, from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### Examples

### Materials and Methods

Tick challenge method The Tick Challenge Method is carried out by applying at least one tick nymph (e.g., I. ricinus) infected with Borrelia (e.g., B. afzelii, strain IS1), to a mouse that is immunized with the multivalent Borrelia vaccine of the invention or pretreated with human sera; and b) applying at least one infected tick nymph to a second mouse that is treated with an appropriate placebo (negative) control, such as buffer or adjuvant alone; and c) comparing the rates of infection in the two mice, generally four to six weeks after challenge. Preferably, groups of mice (e.g. 5-10 mice/groups) are used for each treatment. Assessment of infection status can be done using VISE ELISA on sera and/or qPCR on collected tissues as described in WO2014006226, or using other suitable methods.

Needle challenge method The Needle Challenge Method is carried out for the desired Borrelia strain (e.g., B. burgdorferi, strain N40) by subcutaneously introducing Borrelia at a dose between 20-50× the Infectious Dose (ID)₅₀ to mice that are immunized with the multivalent Borrelia vaccine of the invention or pretreated with human sera, or with an appropriate placebo (negative) control, such as buffer or adjuvant alone and comparing the rates of infection in the challenged mice. The ID₅₀, measured in numbers of bacteria, is defined as the dose at which 50% of untreated challenged mice are infected. The challenge dose can vary widely and is strain-dependent; therefore, the virulence of the strain must first be assessed by challenge experiments for determination of ID₅₀. Four weeks after needle challenge, blood and tissues are collected for readout methods to determine the infection status. The readout methods can be e.g. VIsE ELISA on sera and/or qPCR on collected tissues for identification of Borrelia, as described in WO2014006226, or other methods.

Spirochete antibody binding assay Spirochetes 1×10⁵-1×10⁶ are mixed with an equal volume of 4% paraformaldehyde and incubated for 20 minutes at room temperature in a 96-well plate (Nunclon 96U, Nunc). The plate is centrifuged for 5 minutes at 2,000 g and the supernatant discarded. Cells are washed with 150 μL Hanks' Balanced Salt Solution with 2% BSA (HBSS-B), centrifuged as above, and the supernatant is discarded. Sera are heat inactivated by incubating them at 56° C. for 35 minutes. Heat-inactivated sera are diluted in HBSS-B and sterile filtered by centrifuging at 17,000 g for 3 minutes using Costar spin-X centrifuge tube filters (0.22 μm, Corning, USA). Spirochetes are suspended in 100 μL serum and incubated for 45 minutes at room temperature. Plates are centrifuged for 5 minutes at 2,000 g and the supernatant discarded. The cells are washed once with 150 μL HBSS-B and resuspended in 100 μL HBSS-B with a PE-conjugated secondary antibody diluted 1:150. One microliter of an appropriate secondary antibody (e.g.-PE conjugated anti-mouse or anti-human IgG, available from Beckman Coulter, USA) is added to the cells and incubated at room temperature for 45 minutes in the dark. Spirochetes are washed once with 150 μL HBSS-B and resuspended in 200 μL HBSS containing 10 μg LDS 751 DNA dye and incubated for 10 minutes at room temperature in the dark. The stained spirochetes are pelleted by centrifuging for 5 minutes at 2,000 g and subsequently resuspended in 200 μL HBSS. Labelled spirochetes are measured with a CytoFlex (Beckman Coulter) flow cytometer, gated for LDS 751 positive events.

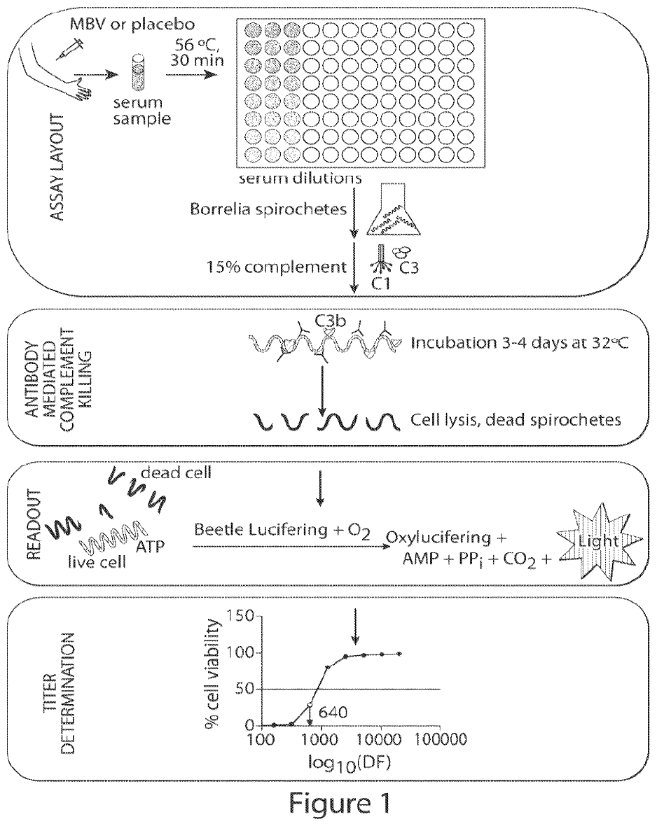

The serum bactericidal assay (SBA) measures vaccine-induced antibodies' ability to kill bacteria in conjunction with exogenously added complement (FIG. 1). Vaccine-induced antibodies recognize surface-exposed antigens on spirochetes and recruit complement, which is activated mostly via the classical pathway, resulting in the formation of membrane attacking complex, bacteriolysis and eventually death of the target bacteria. Serum samples are 2-fold serially diluted with BSK-II medium, subsequently mixed with Borrelia obtained from an overnight culture and guinea pig or baby rabbit complement (source and final concentration depending on the serotype 1-25%) in BSK-II medium, in a white 96-well assay plate. The plate is then incubated at +32° C., 5% CO₂ for 3-6 days (dependent on Borrelia serotype). The amount of live (metabolically active) Borrelia is determined at the end of the assay by measuring ATP dependent light emission with a luciferase-based reaction (BacTiter-Glo, Promega). Human serum pools serve as positive quality controls (QCs), while a commercially available naïve human serum serves as a negative control (NC). To calculate the sample titer, each sample dilution is compared to the corresponding dilution of the negative human serum (NC) for which relative light units are set to 100%. The SBA titer of a test sample is the reciprocal of the highest serum dilution to induce≤50% viability. For Serotype 1, 2, 4, 5, 6 values below the quantitation limit of the SBA (titer of 20) are replaced by titer of 10. For Serotype 3 values below the quantitation limit of the SBA (titer of 160) are replaced by titer of 80.

OspA serotype-specific Human Serum IgG Enzyme-linked immunosorbent assay (ELISA) The IgG geometric mean titers for OspA serotype-specific antibodies were determined by ELISA. Plates were coated with full-length lipidated OspA from ST1, ST2, ST3, ST4, ST5 or ST6. Serial dilutions of trial sera, reference substance (RS) and QC samples were prepared from human serum pool. A blank control was also used. Bound antibodies were detected with human IgG-specific secondary antibody conjugate. OspA-specific IgG concentrations were calculated using SoftMax Pro 5.2 G×P software. Parallel line analysis curves are created individually for each sample tested against the Reference Substance. The relative potency given in these curves is used for calculation of the IgG concentration (U/mL) of the sample compared to the Reference Substance concentration curve. Modifications were made in the ELISA assay from the Phase 1 trial to the Phase 2 trial (see Table A-1). The quantification limit of the ELISA used in the Phase 1 trial (Example 1) was 20 ELISA units (EU)/mL (also referred to herein as U/mL); values <20 EU/mL were replaced by a value of 10 EU/mL. The slightly altered ELISA used in the first Phase 2 trial (Example 2) had a quantification limit of 40 EU/mL; values <40 EU/mL were replaced by a value of 20 EU/mL.

TABLE A-1
Differences in IgG determination (ELISA)
in Phase 1 and Phase 2 clinical trials

| | Phase 1 | Phase 2 |
|---|---|---|
| Coating | His-tagged lipidated full | lipidated full-length OspA |
| antigen | length OspA (0.5 μg/mL) | (0.5 μg/mL) |
| QC | Monoclonal Ab | Polyclonal human serum |
| | | pools |
| Reference | Phase 1 serum pool | New RS prepared from Phase |
| Substance (RS) | | 1 booster samples |
| Lower limit of | 20 U/mL | 40 U/mL |
| quantification | | |
| (LLOQ) | | |
| Seroconversion | Seroconversion rate is | Seroconversion is defined as |
| (SC) | defined as proportion of | change from seronegative to |
| | subjects achieving a ≥4- | seropositive (≥40 U/mL), |
| | fold rise in IgG antibody | or ≥4-fold rise in IgG |
| | titer from Day 0. | antibody titer from Day 0. |

### Example 1. Phase 1 Clinical Trial with Multivalent Borrelia Vaccine

### Phase 1 Clinical Trial Design and Immunogenicity Results

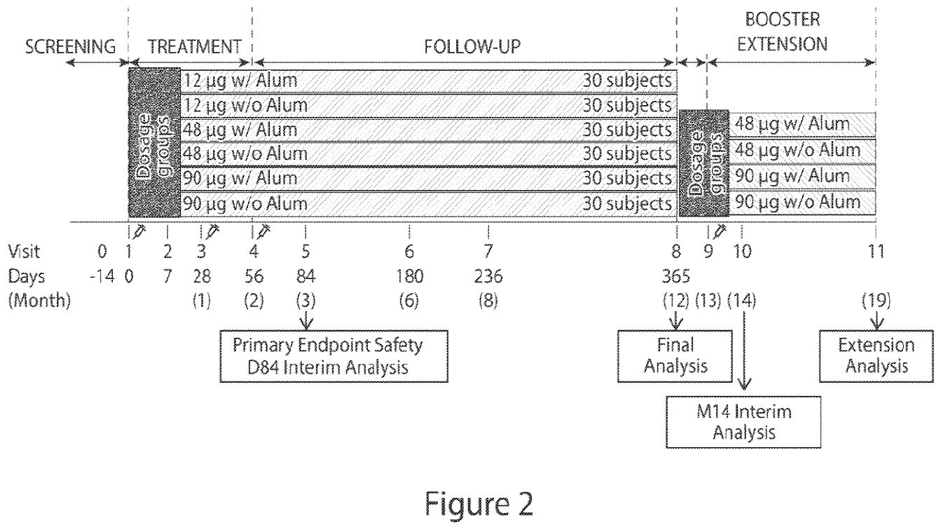

A first-in-human, observer-blinded, partially randomized, multi-center dose escalation Phase I clinical study was undertaken, with the aim to investigate the safety and immunogenicity of different doses of the multimeric Borrelia vaccine (“MBV”) of the invention with and without aluminium hydroxide. The study was conducted in healthy adults aged 18 to 39 years who were baseline seronegative for Borrelia burgdorferi sensu lato (i.e., subjects that were previously not infected with Borrelia). The trial design is shown in FIG. 2. Briefly, the safety characteristics and immunogenicity of three intramuscular vaccinations delivered 28 days apart were evaluated using six different treatments (3 dose levels, each with and without adjuvant) in 179 healthy subjects (i.e., approx. 30 subjects per treatment group). All formulations contained each of the OspA fragment polypeptides (SEQ ID Nos: 1, 2 and 3) at a 1:1:1 weight ratio (e.g.-the low dose (12 μg) contained 4 μg each of the lipidated polypeptides defined by SEQ ID Nos: 1, 2 and 3).

Phase 1 Trial Dose Groups (Vaccinated I.M. d0, d28, d56):
12 μg with Alum (n=29)
12 μg without Alum (n=29)
48 μg with Alum (n=31)
48 μg without Alum (n=29)
90 μg with Alum (n=31)
90 μg without Alum (n=30)

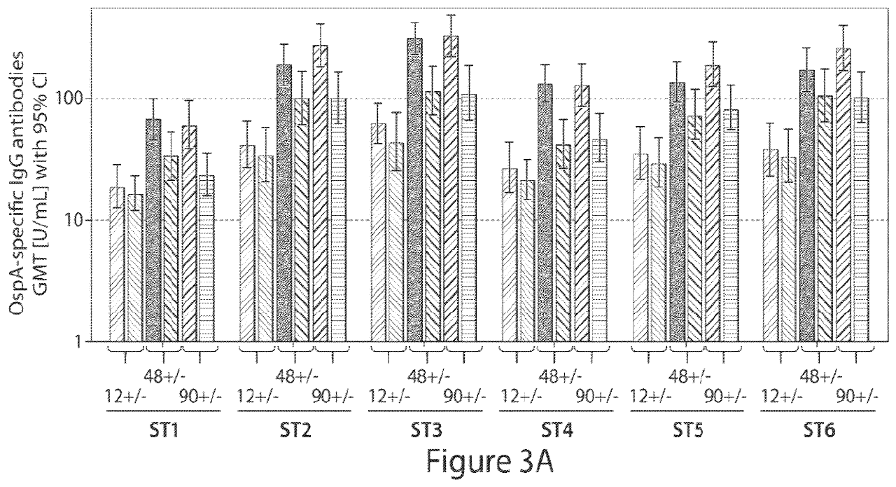

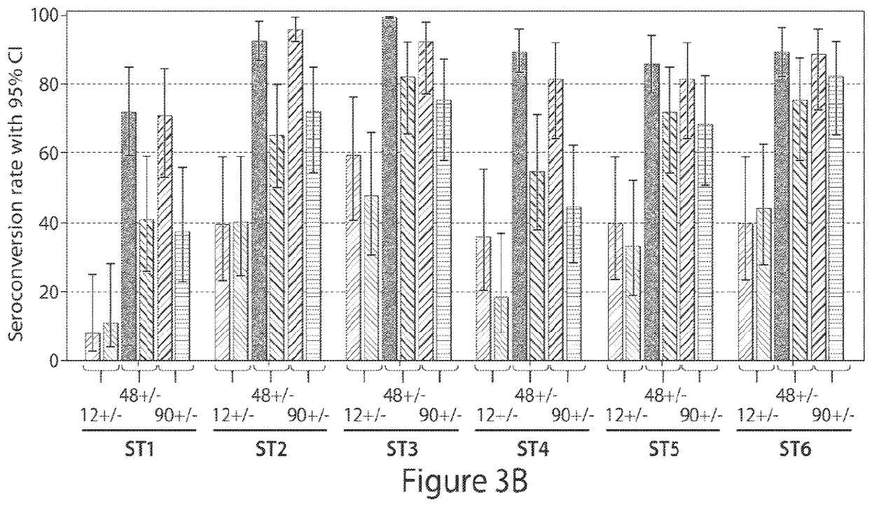

Immunogenicity was assessed in all groups at days 0, 28, 57 84, 180, 236 and 365 with peak antibody titers expected at day 84 (i.e., 4 weeks after the third vaccination, primary endpoint analysis). Geometric mean titers against each OspA serotype at day 84 are shown in FIG. 3A. FIG. 3B shows seroconversion rates (i.e. rate of subjects that seroconvert) for each group, wherein seroconversion is defined as a ≥4-fold increase in IgG titer from baseline.

Booster extension phase On the basis of results from the day 84 analysis, the 48 μg and 90 μg dosage groups with or without adjuvant were selected to receive a booster vaccination at approximately Month 13 with a further 6 months follow-up for safety and antibody persistence. The booster doses administered were the same doses and formulations as for priming. The booster extension part of this study was conducted at one of three study sites only, leading to substantially lower subject number of N=64 subjects in the booster phase.

### Phase 1 Trial Booster Extension Dose Groups (Boosted I.M. Month 13):

48 μg with Alum (n=15)
48 μg without Alum (n=16)
90 μg with Alum (n=16)
90 μg without Alum (n=17)

Immunogenicity was assessed at months 13 (pre-boost), 14 and 19 in the booster extension groups. The objectives of the booster extension were to investigate the safety and immunogenicity of a booster dose of Vaccine A applied approximately one year after the last immunization of the primary schedule up to six months after application of the booster dose. Another use of the booster extension was to generate high titer sera for functional assay development and potential proof-of-concept (PoC) studies. Furthermore, the booster allowed collection of safety and immunogenicity data of a booster dose early in clinical development.

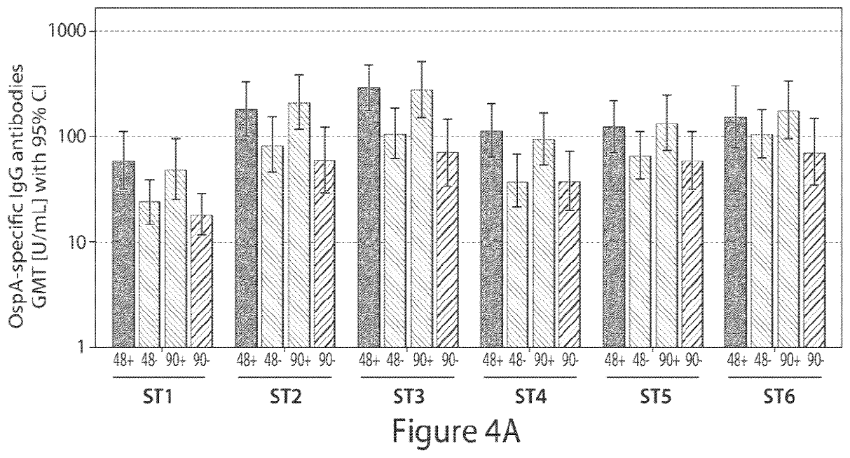

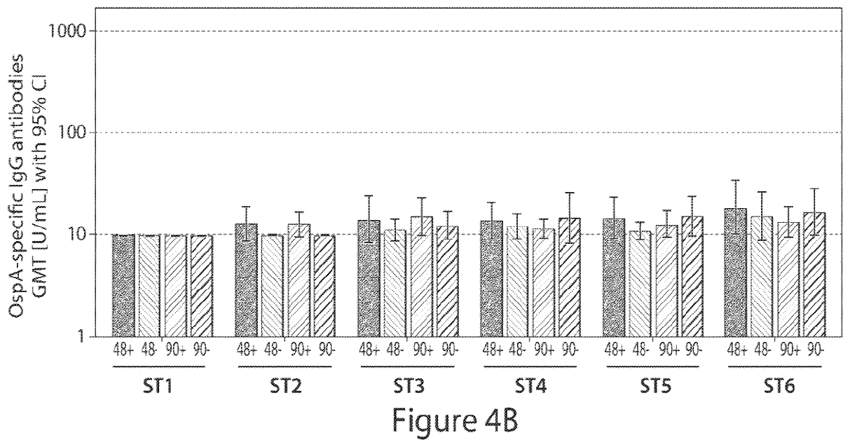

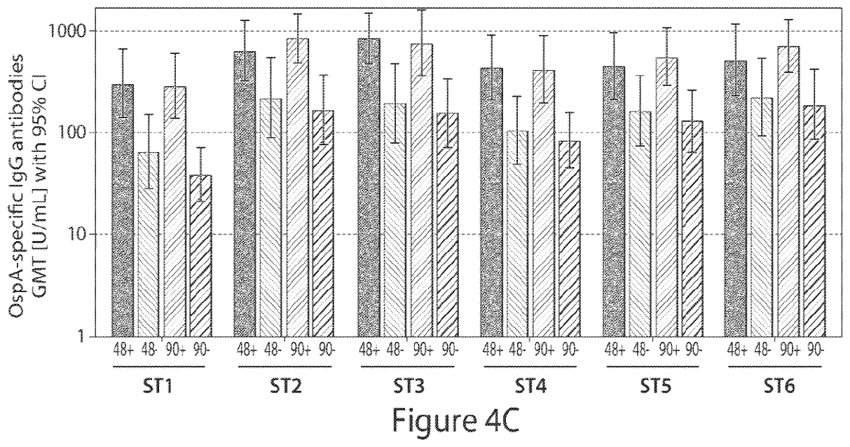

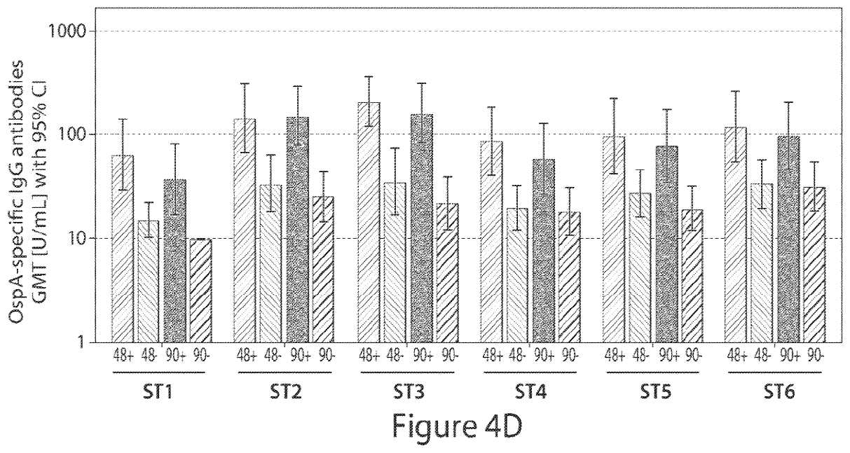

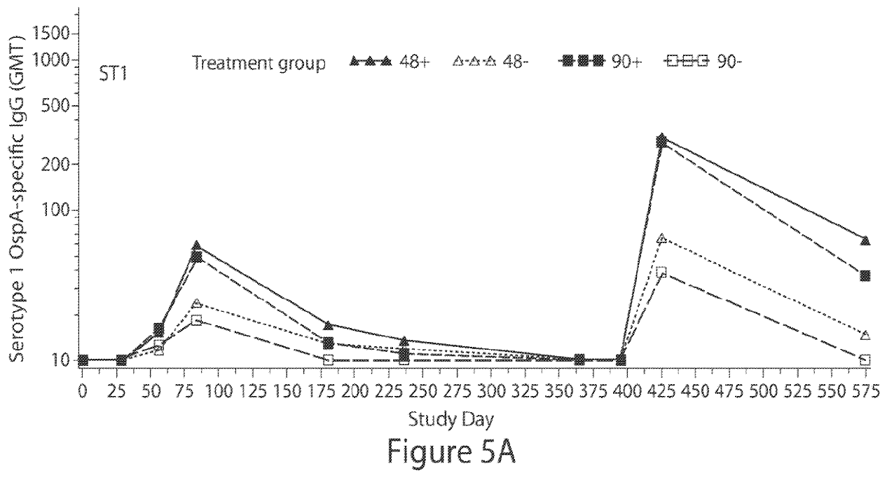

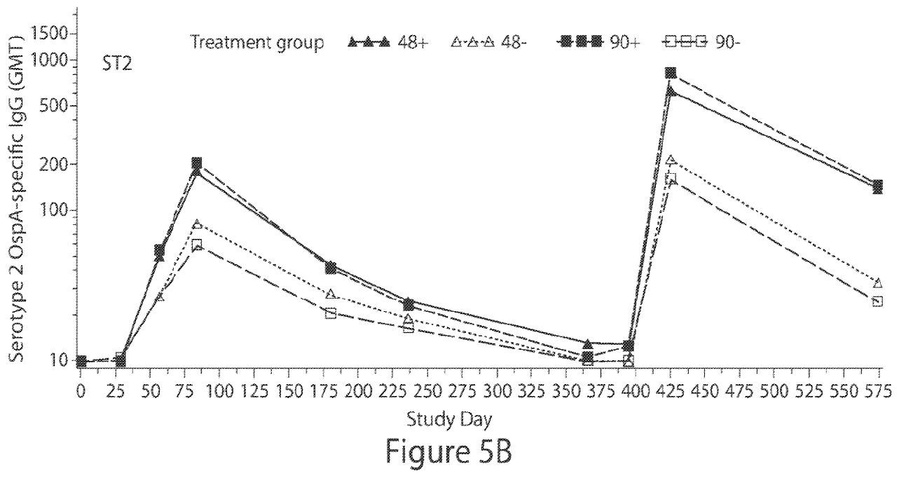

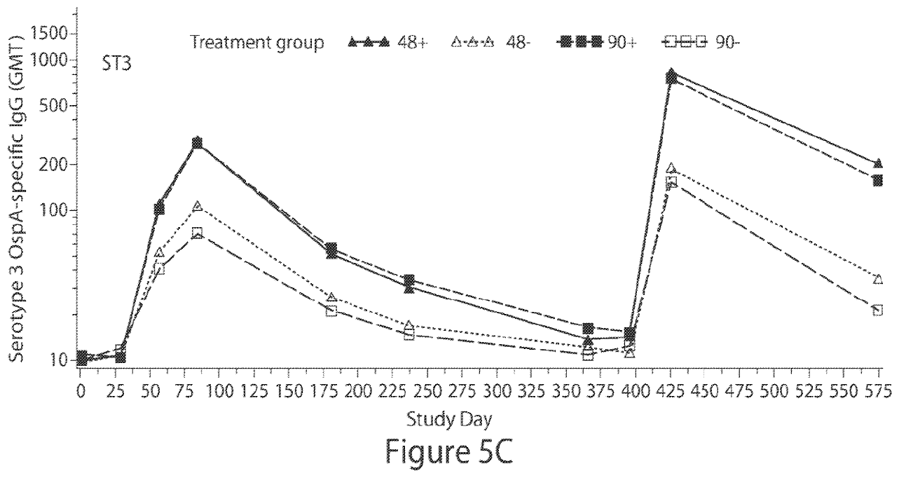

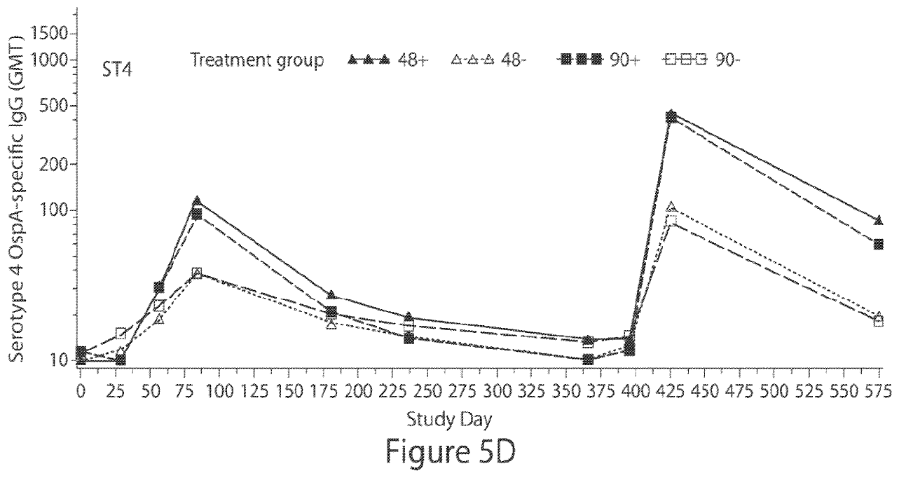

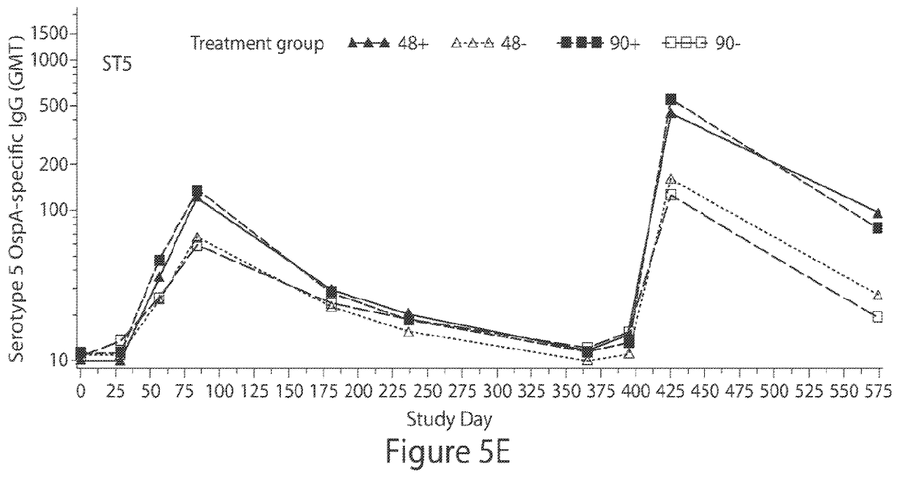

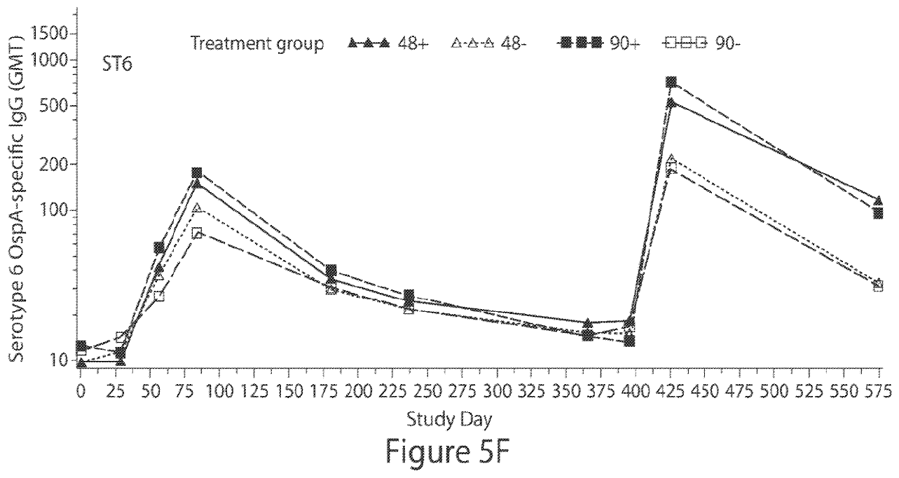

The IgG GMT values of subjects included in the primary analysis of the booster extension phase (Booster PP population) at Day 84 and Months 13, 14 and 19 are shown in FIGS. 4A-D, respectively, and in Table 1 below. Geometric mean fold rise (GMFR) of IgG titer compared to pre-booster time point (month 13) and compared to peak titer after primary vaccination series (day 84) as well as seroconversion rates for day 84, month 13 and month 14 are provided in Table 2 and Table 3, respectively. For comparison of the groups with respect to immune responses to each of the six serotypes over the duration of the entire study, GMT values of the subjects included in the booster extension phase (Booster PP population, i.e., subjects enrolled in booster extension phase and received correct booster dose at correct timing according to protocol) over the duration of the study are provided in FIGS. 5A-F.

TABLE 1
GMTs of serotype specific IgG titer on Day 84, M 13, M 14 and M 19, Booster PP population.

| | GMT [U/mL] | | | | | | | | | | | | | | | |
|---|---|---|---|---|---|---|---|---|---|---|---|---|---|---|---|---|
| | 48 μg + Alum | | | | 48 μg − Alum | | | | 90 μg + Alum | | | | 90 μg − Alum | | | |
| | Timepoint | | | | | | | | | | | | | | | |
| | D 84 | M 13 | M 14 | M 19 | D 84 | M 13 | M 14 | M 19 | D 84 | M 13 | M 14 | M 19 | D 84 | M 13 | M 14 | M 19 |
| ST1 | 60 | 10 | 305 | 64 | 24 | 10 | 66 | 15 | 49 | 10 | 286 | 37 | 18 | 10 | 39 | 10 |
| ST2 | 185 | 12 | 640 | 144 | 84 | 10 | 221 | 34 | 212 | 13 | 840 | 152 | 60 | 10 | 167 | 25 |
| ST3 | 292 | 13 | 832 | 208 | 109 | 12 | 194 | 35 | 283 | 15 | 763 | 161 | 72 | 12 | 156 | 22 |
| ST4 | 116 | 12 | 436 | 87 | 38 | 12 | 105 | 20 | 95 | 12 | 416 | 59 | 38 | 13 | 84 | 18 |
| ST5 | 125 | 13 | 455 | 96 | 67 | 11 | 164 | 28 | 136 | 13 | 558 | 78 | 59 | 14 | 129 | 19 |
| ST6 | 154 | 18 | 523 | 119 | 106 | 15 | 223 | 34 | 180 | 15 | 713 | 98 | 72 | 15 | 189 | 32 |

TABLE 2
Geometric mean fold rise (GMFR) of serotype specific IgG titer, on Month 14 and Month 19, compared to Month
13 (pre-boost) and Day 84 (peak antibody titer after primary immunization series), Booster PP population.

| | GMFR | | | | | | | | | | | | | | | |
|---|---|---|---|---|---|---|---|---|---|---|---|---|---|---|---|---|
| | 48 μg + Alum | | | | 48 μg − Alum | | | | 90 μg + Alum | | | | 90 μg − Alum | | | |
| | Month | | | | | | | | | | | | | | | |
| | M 14 | M 14 | M 19 | M 19 | M 14 | M 14 | M 19 | M 19 | M 14 | M 14 | M 19 | M 19 | M 14 | M 14 | M 19 | M 19 |
| | Compared to timepoint | | | | | | | | | | | | | | | |
| | M 13 | D 84 | M 13 | D 84 | M 13 | D 84 | M 13 | D 84 | M 13 | D 84 | M 13 | D 84 | M 13 | D 84 | M 13 | D 84 |
| ST1 | 31 | 5 | 6 | 1.1 | 7 | 2 | 2 | 0.6 | 29 | 6 | 4 | 0.7 | 4 | 2 | 1 | 0.5 |
| ST2 | 54 | 4 | 12 | 0.8 | 22 | 2 | 3 | 0.4 | 67 | 4 | 12 | 0.7 | 17 | 3 | 3 | 0.4 |
| ST3 | 66 | 3 | 17 | 0.7 | 15 | 2 | 3 | 0.3 | 52 | 3 | 11 | 0.6 | 13 | 2 | 2 | 0.3 |
| ST4 | 35 | 4 | 7 | 0.7 | 10 | 3 | 2 | 0.5 | 36 | 4 | 5 | 0.6 | 6 | 2 | 1 | 0.5 |
| ST5 | 35 | 4 | 7 | 0.8 | 15 | 2 | 3 | 0.4 | 44 | 4 | 6 | 0.6 | 9 | 2 | 1 | 0.3 |
| ST6 | 30 | 3 | 7 | 0.8 | 17 | 2 | 3 | 0.3 | 49 | 4 | 7 | 0.5 | 13 | 3 | 2 | 0.5 |

TABLE 3
Seroconversion Rates (SCRs) per serotype at Day 84, M 13, M 14 and M 19, Booster PP population.

| | SCR [%] | | | | | | | | | | | | | | | |
|---|---|---|---|---|---|---|---|---|---|---|---|---|---|---|---|---|
| | 48 μg + Alum | | | | 48 μg − Alum | | | | 90 μg + Alum | | | | 90 μg − Alum | | | |
| | Timepoint | | | | | | | | | | | | | | | |
| | D 84 | M 13 | M 14 | M 19 | D 84 | M 13 | M 14 | M 19 | D 84 | M 13 | M 14 | M 19 | D 84 | M 13 | M 14 | M 19 |
| ST1 | 67 | 0 | 93 | 67 | 31 | 0 | 57 | 20 | 63 | 0 | 94 | 50 | 29 | 0 | 47 | 0 |
| ST2 | 87 | 0 | 100 | 87 | 69 | 0 | 86 | 47 | 94 | 0 | 100 | 88 | 59 | 0 | 82 | 38 |
| ST3 | 100 | 13 | 100 | 100 | 88 | 13 | 86 | 53 | 94 | 13 | 94 | 88 | 65 | 6 | 82 | 38 |
| ST4 | 80 | 7 | 93 | 73 | 56 | 0 | 79 | 33 | 75 | 0 | 88 | 63 | 29 | 6 | 82 | 19 |
| ST5 | 80 | 13 | 93 | 73 | 75 | 0 | 86 | 33 | 75 | 6 | 100 | 69 | 59 | 12 | 82 | 25 |
| ST6 | 80 | 13 | 93 | 80 | 81 | 13 | 86 | 47 | 88 | 13 | 100 | 75 | 71 | 12 | 82 | 31 |

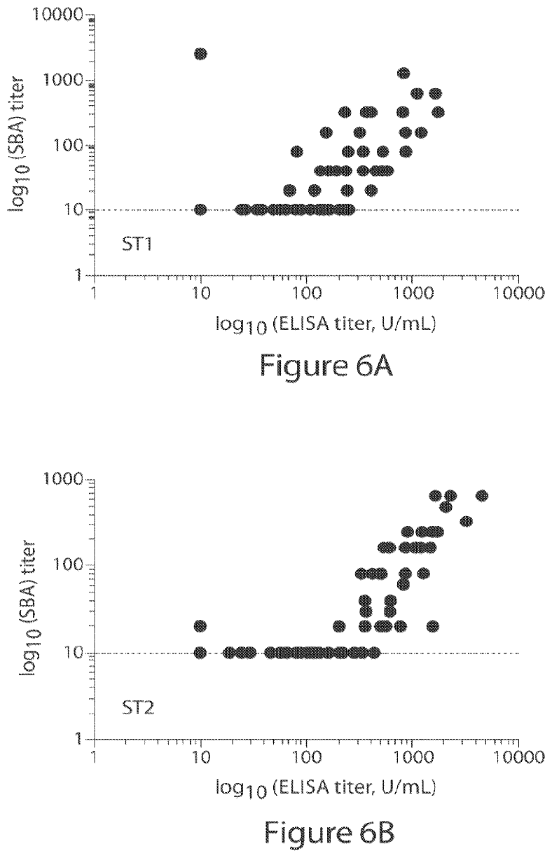

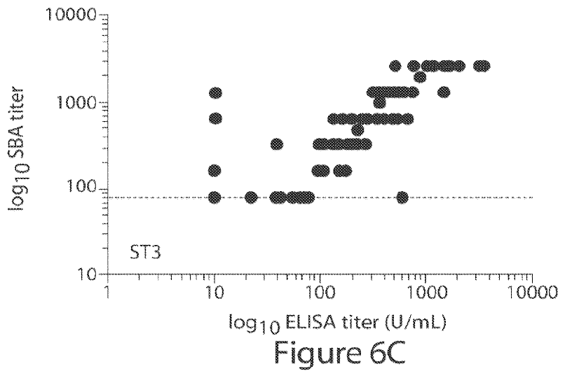

Correlation of ELISA and SBA Titers in Phase 1 serum samples The serotype-specific serum bactericidal assay (SBA-see Materials and Methods), an in vitro correlate of protection for clinical samples, yielded values that correlated closely with ELISA values for serotypes 1, 2 and 3 in individual subject sera, as shown in FIG. 6A, B and C, respectively.

In summary, serotype specific IgG antibodies were induced with all doses and formulation investigated in phase 1, with higher immunogenicity observed for the higher dose groups and for the adjuvanted formulations. A booster dose applied approx. one year after finalization of the primary vaccination series resulted in a substantial booster response (i.e., IgG titers were substantially higher at 4 weeks after booster dose as compared to 4 weeks after completion of the primary immunization). for all six OspA serotypes and were sustained at higher levels through month 19 (Table 3). As sustained circulating antibody levels are of high importance for the efficacy of OspA-based vaccines, a further dose increase and alternative schedules were introduced in Phase 2, with the aim to induce an earlier, higher and more durable immune response.

### Example 2. Phase 2 Clinical Trial with Multivalent Borrelia Vaccine Investigating Higher Doses of the Vaccine

Phase 2 Clinical Trial Design and Summary of Preliminary Descriptive Immunogenicity Data from the Run-In Phase

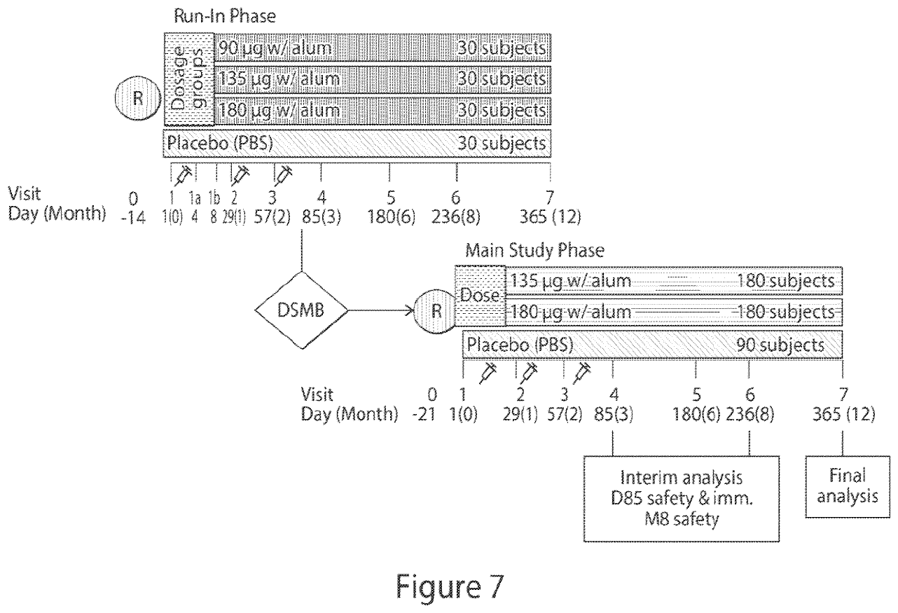

An observer-blinded, randomized, placebo controlled, multicenter phase 2 study is currently ongoing with the aim to investigate the safety and immunogenicity of a range of higher doses of the multimeric Borrelia vaccine adjuvanted with aluminium hydroxide in healthy adults aged 18 to 65 years. Subjects who were baseline seronegative for Borrelia burgdorferi sensu lato (i.e., subjects that were previously not infected with Borrelia) as well as subjects who were baseline seropositive for Borrelia burgdorferi sensu lato (i.e., subjects that were previously infected with Borrelia) were enrolled in this study. The study consists of a safety run-in phase (ages 18-40, N=120 subjects) in order to investigate the safety of the three dose levels of 90 μg, 135 μg and 180 μg multimeric Borrelia vaccine (“MBV”) of the invention with and without alum initially in a smaller population prior to initiating the main study phase (ages 18-65, N=450 subjects) (see FIG. 7) with the two higher dose groups of 135 μg and 180 μg. Subject received three I.M. vaccinations one month apart (i.e., Month 0-1-2). In the main study phase subjects were enrolled in two age groups (18-49 years and 50-65 years) in a ratio of approx. 2:1. The main objectives of the Phase 2 trial are to assess the immunogenicity and safety of the multimeric Borrelia vaccine in healthy adults aged 18-65 years with the higher dose levels as compared to the first-in-human trial and to determine the optimal dose of the vaccine. All formulations contained each of the OspA fragment polypeptides (SEQ ID Nos: 1, 2 and 3) at a 1:1:1 weight ratio (e.g.-the low dose (90 μg) contained 30 μg each of the lipidated polypeptides defined by SEQ ID Nos: 1, 2 and 3, the high dose (180 μg) contained 60 μg each of the lipidated polypeptides defined by SEQ ID Nos: 1, 2 and 3).

Run-in phase A total of 120 healthy subjects aged 18 to 40 years were to be enrolled into four treatment groups (vaccinated I.M. d1, d29, d57): 90 μg of the multimeric Borrelia vaccine (“MBV”) of the invention with Alum, 135 μg the MBV with Alum, 180 μg MBV with Alum, Placebo (PBS), approximately 30 subjects per treatment group. After Data Safety Monitoring Board (DSMB) review of safety data up to Day 85, these two higher dose groups (i.e., 135 μg and 180 μg) were selected for further investigation in the Main Study phase. Sera are collected at Day 1, 29, 57, 85 (primary immunogenicity analysis), 180, 236 and 365 and will be assessed for immunogenicity by ELISA and SBA (selected time points in a representative subset of subjects).

A preliminary descriptive analysis of immunogenicity data from the run-in phase revealed that the multivalent Borrelia vaccine was immunogenic at all dose levels tested in the run-in phase. The preliminary GMT titers (ELISA) in the 90 μg w/alum group were comparable to those obtained in the Phase 1 trial at the same dose level. A dose-response was observed, with the lowest IgG titers in the 90 μg dose group and the highest titers in the 180 μg dose group for all serotypes. The dose increase compared with Phase 1 resulted in an increased peak immune response after primary immunization, as anticipated. However antibody levels declined relatively substantially up to Day 180 in all dose groups. Given the observed decline in antibody titers after the primary series in the Phase 2 run-in phase, future development of the multimeric Borrelia vaccine should consider a broader Month 0-2-6 schedule. Based on experience with other vaccines, this is expected to result in even higher peak titers and importantly better persistence and may also improve antibody quality.

Main phase A total of 450 healthy subjects aged 18 to 65 years were to be enrolled into three treatment groups (vaccinated I.M D1, d29, d57): 135 μg with Alum (n=approx. 180), 180 μg with Alum (n=approx. 180), Placebo (PBS) (n=approx. 90). A first data analysis was performed which included pooled immunogenicity data (GMTs of OspA serotype specific IgG by ELISA) from Day 85 of the main study phase and run in phase. Functionality of antibodies at Day 85 was assessed using a Serum Bactericidal Assay (SBA). The data confirmed the preliminary data analysis of the run-in phase in terms of higher antibody titers after completion of the primary immunization series, i.e., higher antibody levels were achieved at Day 85 with the 135 μg (GMTs range from 101.1 [ST1] to 282.2 [ST3]) and the 180 μg dose groups (GMTs range from 115.8 [ST1] to 308.6 [ST3]) as compared to the 90 μg dose group investigated in the run in phase (GMTs range from 74.3 [ST1] to 267.4 [ST3]). Data reveal that older adults (age group 50-65 years) which are one of the high risk groups for acquiring Lyme disease have also shown an encouraging immune response, without statistical significant differences between the two age groups. Refer to Table 4 for a summary of immunogenicity data.

Furthermore, the Borrelia vaccine as set out above (OspA fragment polypeptides (SEQ ID Nos: 1, 2 and 3) at a 1:1:1 weight ratio (e.g.-the low dose (90 μg) contained 30 μg each of the lipidated polypeptides defined by SEQ ID Nos: 1, 2 and 3, the high dose (180 μg) contained 60 μg each of the lipidated polypeptides defined by SEQ ID Nos: 1, 2 and 3) was immunogenic across all dose groups tested. Higher doses used in this trial elicited higher antibody responses across all serotypes compared to Phase 1. Seroconversion rates (SCR) in the highest dose ranged from 81.5% (ST1) to 95.8% (ST2). In the age group comparable to the age group investigated in Phase 1 (18-49 yrs) SCRs ranged from 85.6% to 97% (previous study: 71.4% to 96.4%). The immunological response in older adults, one of the main target groups for a Lyme vaccine, is particularly encouraging. Results did not indicate that prior exposure to Lyme (sero-positivity) has an impact on immunogenicity or safety.

Said Borrelia vaccine was generally safe across all dose and age groups tested. No related Serious Adverse Events (SAEs) were observed with Borrelia vaccine in this study in any treatment group. Reactogenicity decreased with subsequent vaccinations.

Overall the tolerability profile including rates of fever appeared to be comparable to other lipidated recombinant vaccines or lipid-containing formulations.

TABLE 4
Day 85 GMT first Phase 2 study (schedule Month 0-1-2), PP population

| | Run-In Phase | Subjects from Run in Phase and Main Study Phase | | | | | |
|---|---|---|---|---|---|---|---|
| | 90 μg | 135 μg | | | 180 μg | | |
| | 18-40 | 18-49 | 50-65 | 18-65 | 18-49 | 50-65 | 18-65 |
| | years | years | years | years | years | years | years |
| | N = 26 | N = 129 | N = 59 | N = 188 | N = 132 | N = 57 | N = 189 |
| | [EU/mL] | [EU/mL] | [EU/mL] | [EU/mL] | [EU/mL] | [EU/mL] | [EU/mL] |
| ST1 | 74.3 | 103.8 | 95.4 | 101.1 | 129.6 | 89.2 | 115.8 |
| ST2 | 180.9 | 292.7 | 245.2 | 276.9 | 322.1 | 265.0 | 303.7 |
| ST3 | 267.4 | 293.4 | 259.1 | 282.2 | 334.2 | 256.5 | 308.6 |
| ST4 | 117.0 | 175.1 | 158.5 | 169.7 | 206.8 | 158.1 | 190.7 |
| ST5 | 118.3 | 178.8 | 165.8 | 174.6 | 218.3 | 162.1 | 199.6 |
| ST6 | 115.6 | 184.4 | 178.5 | 182.6 | 225.6 | 174.3 | 208.7 |

Seroconversion rates (SCRs) at Day 85 are depicted in Table 12. In the 90 μg dose, SCRs ranged from 65.4% (ST1) to 96.2 (ST3), in the 135 μg dose, SCRs ranged from 80.9% (ST1) to 98.4 (ST2) and in the 180 μg group SCRs ranged from 81.5% (ST1) to 95.8% (ST3).

TABLE 12
Day 85 SCRs (ELISA) first Phase 2 study
(schedule Month 0-1-2), PP population

| | 90 μg | 135 μg | 180 μg |
|---|---|---|---|
| | N = 29 | N = 188 | N = 189 |
| | (%) | (%) | (%) |
| ST1 | 65.4 | 80.9 | 81.5 |
| ST2 | 88.5 | 98.4 | 95.2 |
| ST3 | 96.2 | 97.3 | 95.8 |
| ST4 | 80.8 | 94.1 | 93.7 |
| ST5 | 80.8 | 91.0 | 90.5 |
| ST6 | 80.8 | 93.1 | 91.5 |

Functionality of antibodies has been demonstrated for all serotypes using a serum bactericidal assay (SBA-see Materials and Methods). GMTs at Day 85 are summarized in Table 13.

TABLE 13
GMTs for functional antibodies as measured
by SBA on Day 85 (schedule Month 0-1-2).

| | 135 μg | 180 μg |
|---|---|---|
| | N = 84 | N = 86 |
| | SBA Titer | SBA Titer |
| ST1 | 17.5 | 21.4 |
| ST2 | 14.9 | 17.6 |
| ST3 | 737.6 | 700.1 |
| ST4 | 21.9 | 25.8 |
| ST5 | 23.2 | 24.4 |
| ST6 | 100.2 | 152.4 |

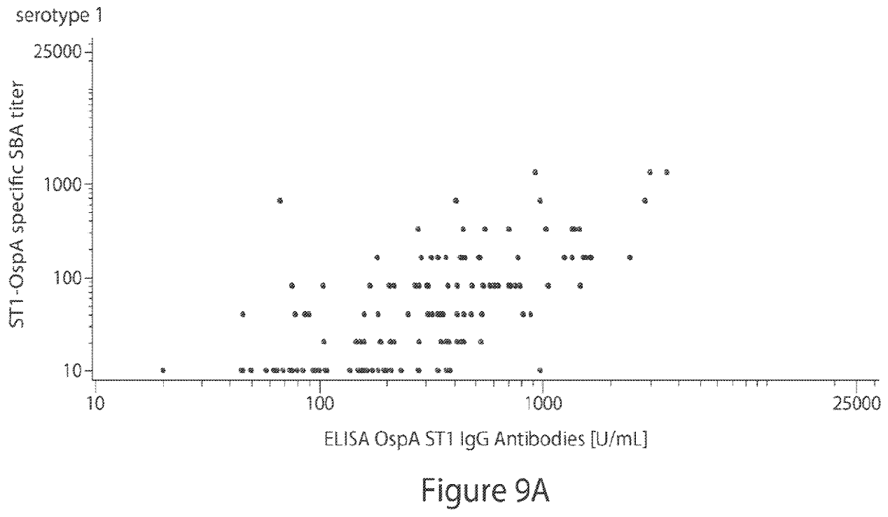

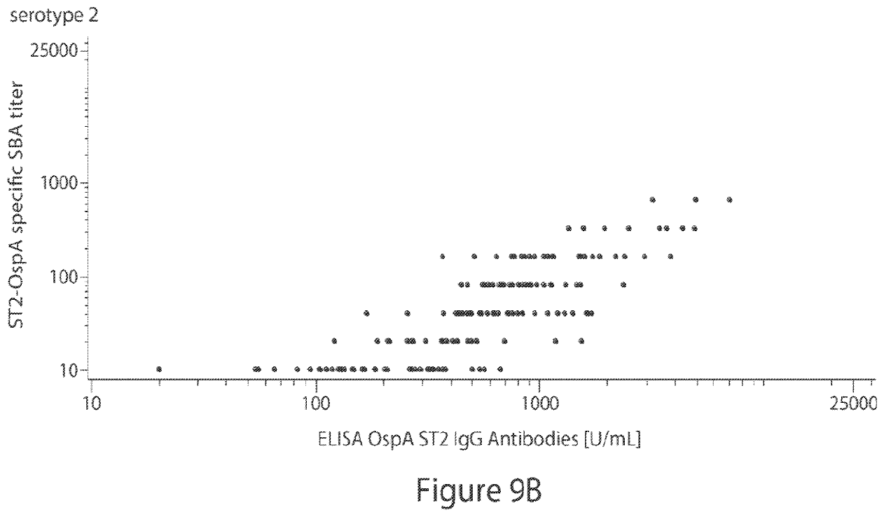

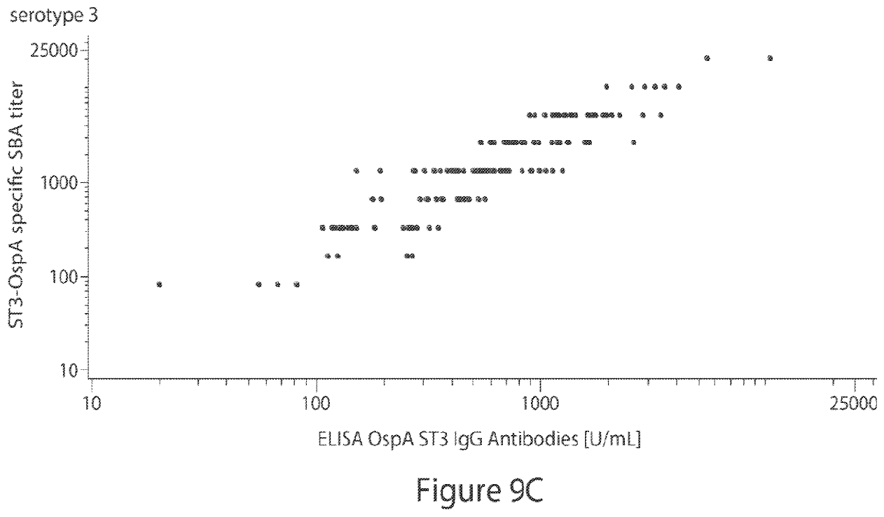

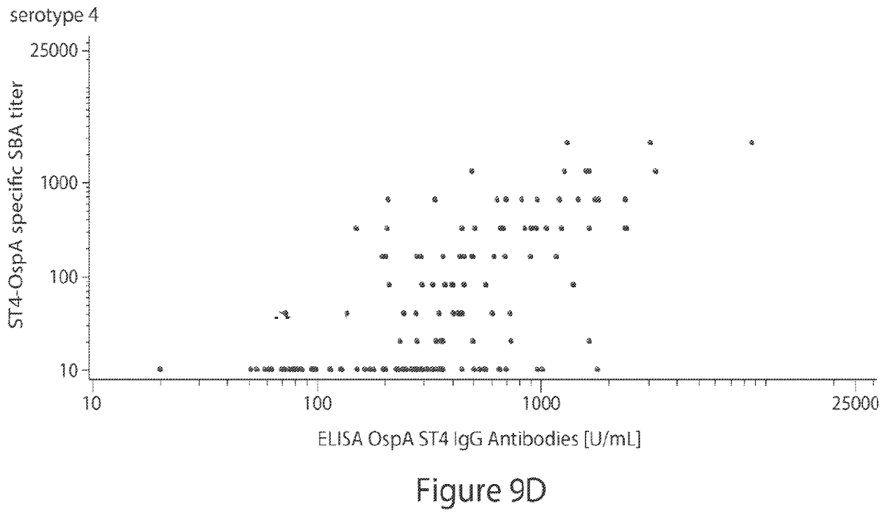

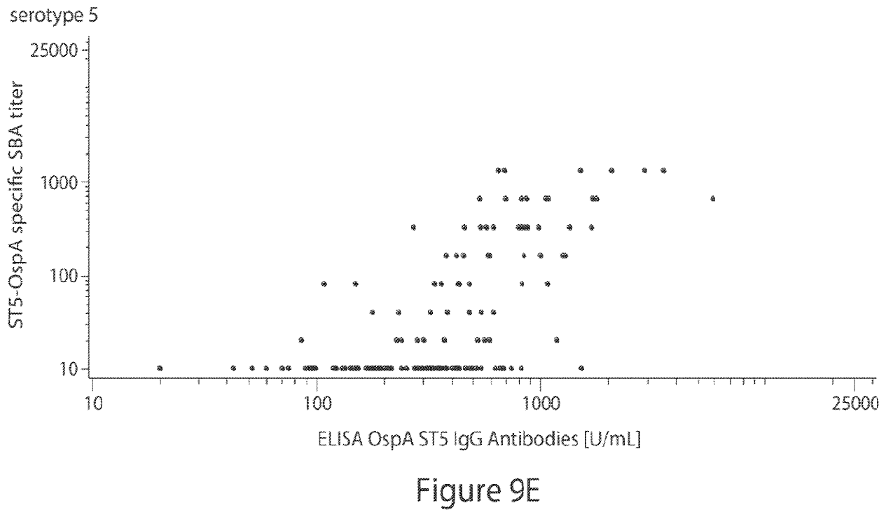

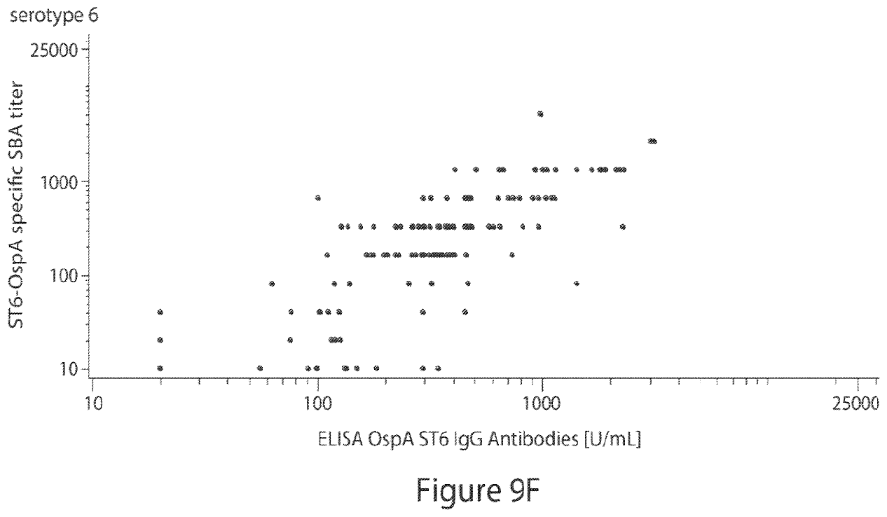

ELISA titer correlated significantly with respective SBA titer for each OspA serotype and individual subject sera, as shown in FIGS. 9A-9F. Spearman correlation coefficients ranged from 0.5737, p-value <0.0001, for serotype 4 to 0.9030, p-value <0.0001, for serotype 3.

### Example 3. Phase 2 Clinical Trial with Multivalent Borrelia Vaccine Investigating Higher Doses of the Vaccine and a Broader Immunization Schedule

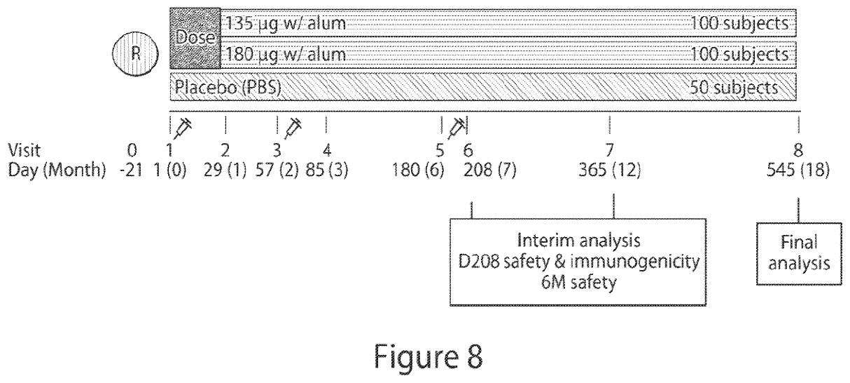

Overall, 246 healthy subjects aged 18 to 65 years were enrolled in this study, an observer-blinded, randomized, placebo controlled, multicenter phase 2 study investigating the same dose groups as tested in example 2 at an alternative, broader immunization schedule. Subjects received three I.M. immunizations of 135 μg or 180 μg of the multimeric Borrelia vaccine formulated w/alum (approx. 100 subjects each) or placebo (approx. 50 subjects) at Month 0, 2 and 6 (Days 1-57-180) (i.e. OspA fragment polypeptides (SEQ ID Nos: 1, 2 and 3) at a 1:1:1 weight ratio (e.g.-the low dose (135 μg) contained 45 μg each of the lipidated polypeptides defined by SEQ ID Nos: 1, 2 and 3, the high dose (180 μg) contained 60 μg each of the lipidated polypeptides defined by SEQ ID Nos: 1, 2 and 3). Subjects were enrolled in two age groups (18-49 years and 50-65 years) in a ratio of approx. 2:1. Also subjects who were baseline seronegative for Borrelia burgdorferi sensu lato (i.e., subjects that were previously not infected with Borrelia) as well as subjects who were baseline seropositive for Borrelia burgdorferi sensu lato (i.e., subjects that were previously infected with Borrelia) were enrolled in this study. The main objectives of the Phase 2 trial are to assess the immunogenicity and safety of the multimeric Borrelia vaccine in healthy adults aged 18-65 years with the higher dose levels as compared to the first-in-human trial and applying a broader immunization schedule as used in the examples above. Together with results obtained of example 2, data will be used to determine the optimal dose and schedule of the vaccine for late stage clinical development. The trial design is shown in FIG. 8.

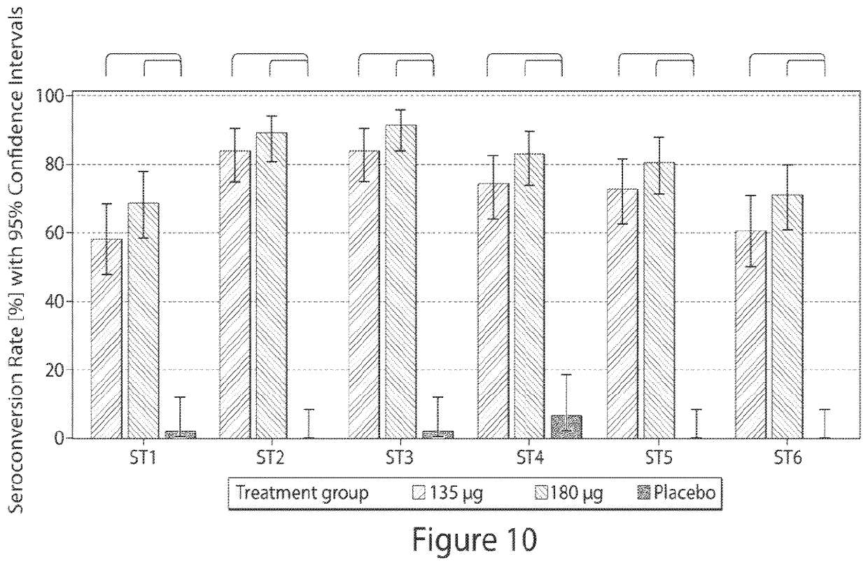

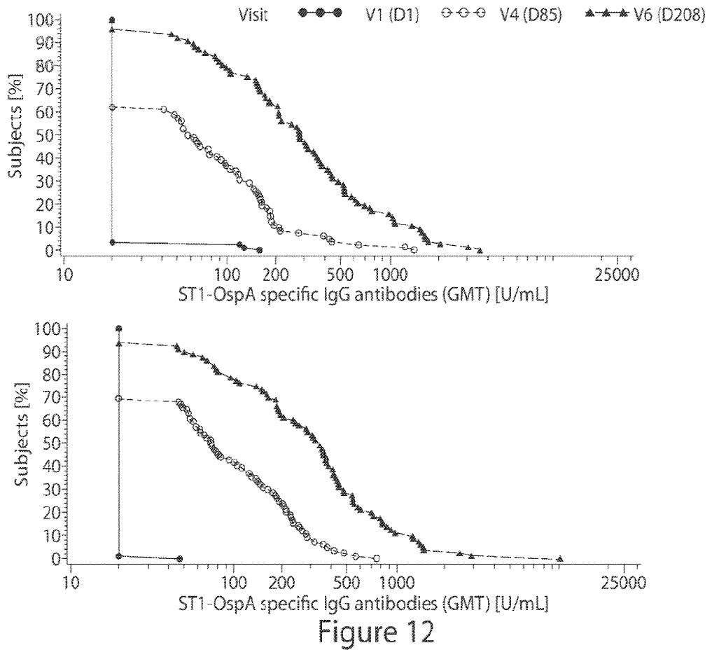

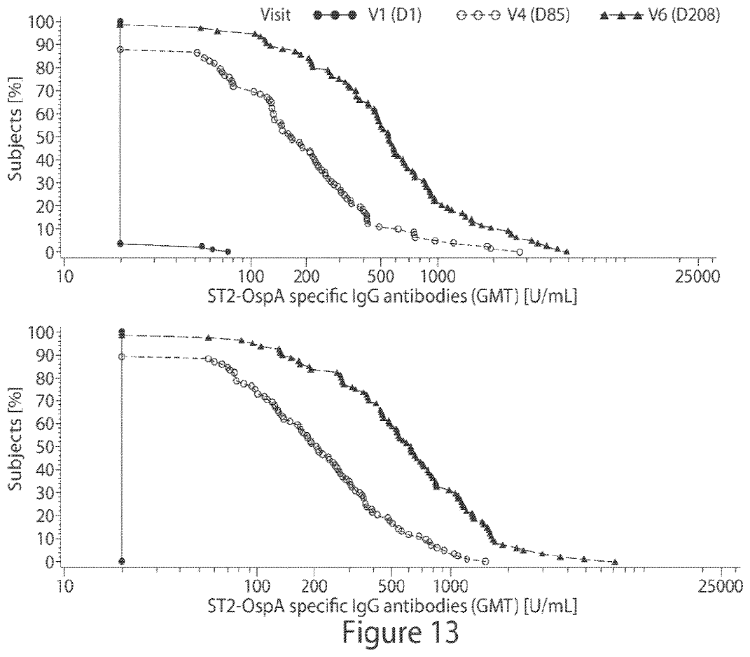

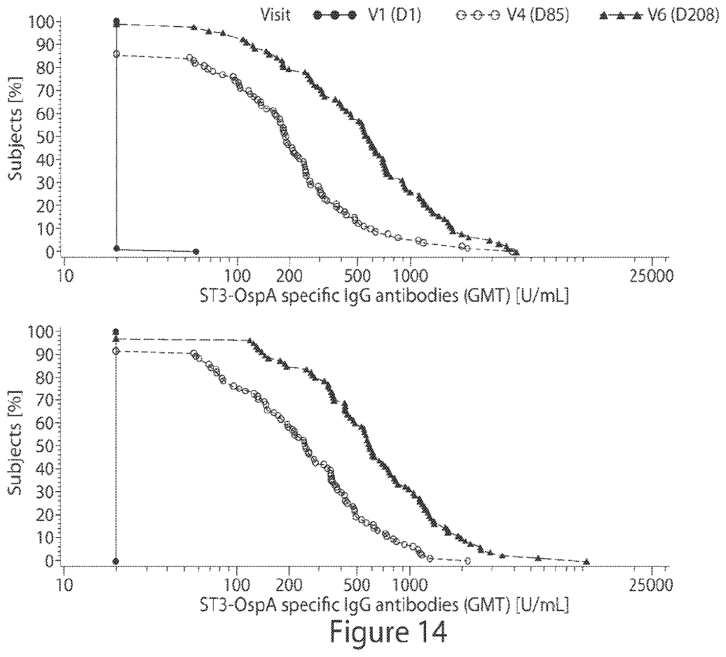

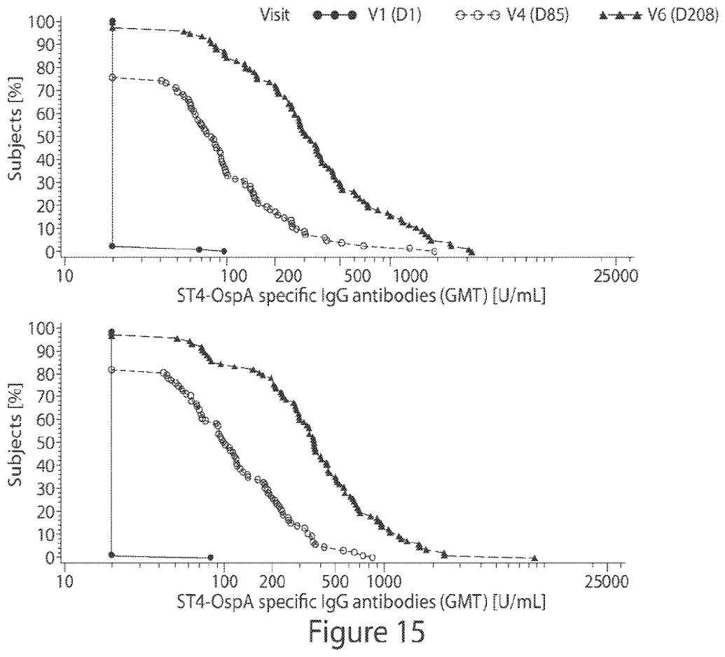

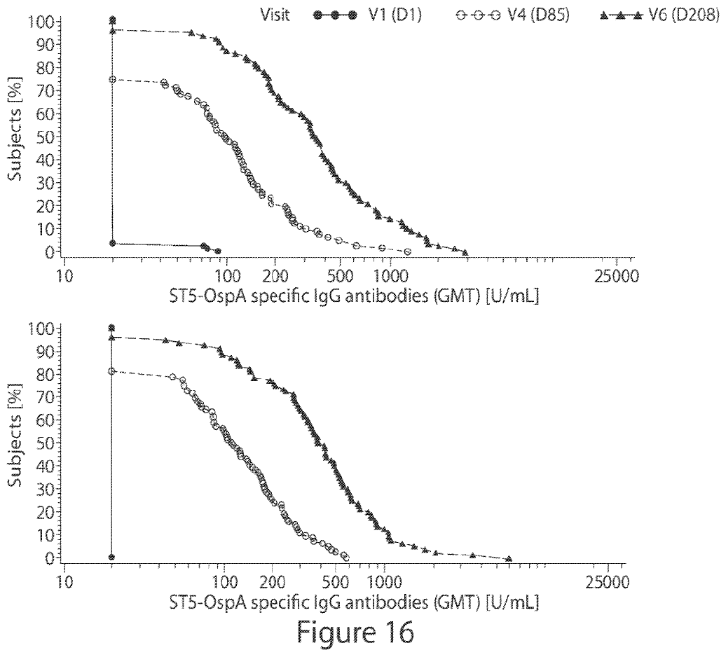

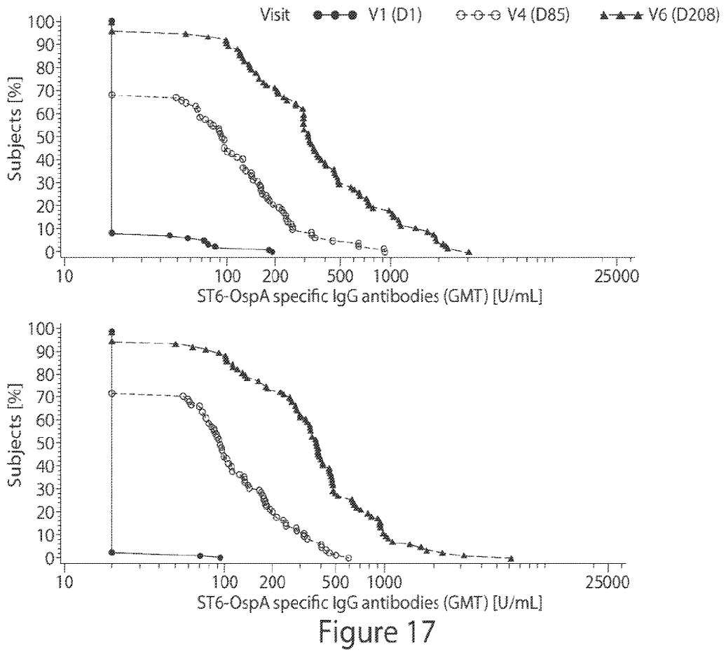

Sera are collected at Day 1, 29, 57, 85, 180, 208 (primary immunogenicity analysis), 365 and 545 and will be assessed for immunogenicity by ELISA and SBA (selected time points). At Day 85 (i.e. after the second vaccination), antibody levels (GMTs) in the 135 μg dose group ranged from 64.1 [ST1] to 166.4 [ST3] and in the 180 μg dose group ranged from 75.2 [ST1] to 217.7 [ST3]. Table 10 provides an overview of Geometric Mean Titers (GMTs) for the second Phase 2 study and Table 11, FIG. 10 provides an overview of seroconversion rate for the second Phase 2 study for Day 85. See also FIGS. 12-17.

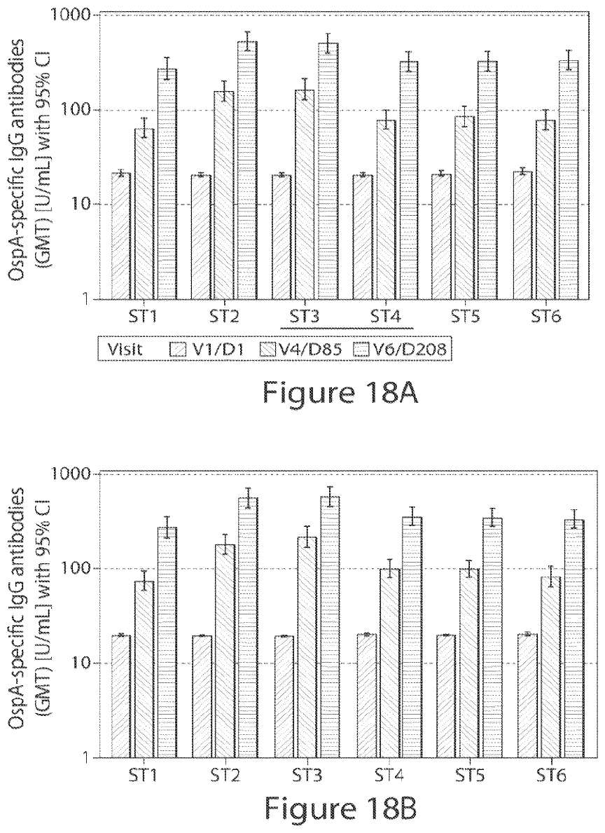

Overall, onset of immune response seems to be somewhat faster with the higher 180 μg dose, i.e. GMTs at Day 85 are higher with the 180 μg dose group compared with the 135 μg group. Refer to FIGS. 18A and 18B for comparison of GMTs between the 135 μg and the 180 μg treatment group and to Table 10 for GMT values at Day 85.

TABLE 10
Day 85 GMT second Phase 2 study (schedule
Month 0-2-6), PP population

| | 135 μg + Alum | 180 μg + Alum | Placebo | |
|---|---|---|---|---|
| | N = 84 | N = 86 | N = 43 | |
| | [EU/mL] | [EU/mL] | [EU/mL] | p-value |
| | [95% CI] | [95% CI] | [95% CI] | (Overall) |
| ST1 | 64.1 | 75.2 | 21.7 | <.0001 |
| | [50.2, 81.7] | [59.6, 94.9] | [19.7, 23.8] | |
| ST2 | 160.8 | 183.5 | 20.0 | <.0001 |
| | [125.4, 206.3] | [144.9, 232.4] | [20.0, 20.0] | |
| ST3 | 166.4 | 217.7 | 20.7 | <.0001 |
| | [128.3, 215.9] | [171.7, 275.9] | [19.3, 22.2] | |
| ST4 | 78.7 | 100.8 | 21.6 | <.0001 |
| | [62.5, 99.0] | [81.2, 125.0] | [20.0, 23.3] | |
| ST5 | 86.8 | 100.2 | 20.0 | <.0001 |
| | [68.5, 110.0] | [81.0, 124.1] | [20.0, 20.0] | |
| ST6 | 79.5 | 84.3 | 20.7 | <.0001 |
| | [62.4, 101.3] | [67.4, 105.4] | [19.3, 22.1] | |

TABLE 11
Day 85 SCRs second Phase 2 study (schedule
Month 0-2-6), PP population

| | 135 μg + Alum | 180 μg + Alum | Placebo | |
|---|---|---|---|---|
| | N = 84 | N = 86 | N = 43 | |
| | n/Nm (%) | n/Nm (%) | n/Nm (%) | p-value |
| | [95% CI] | [95% CI] | [95% CI] | (Overall) |
| ST1 | 48/82 (58.5) | 58/84 (69.0) | 1/43 (2.3) | <.0001 |
| | [47.7, 68.6] | [58.5, 77.9] | [0.4, 12.1] | |
| ST2 | 69/82 (84.1) | 75/84 (89.3) | 0/43 (0.0) | <.0001 |
| | [74.7, 90.5] | [80.9, 94.3] | [0.0, 8.2] | |
| ST3 | 69/82 (84.1) | 77/84 (91.7) | 1/43 (2.3) | <.0001 |
| | [74.7, 90.5] | [83.8, 95.9] | [0.4, 12.1] | |
| ST4 | 61/82 (74.4) | 70/84 (83.3) | 3/43 (7.0) | <.0001 |
| | [64.0, 82.6] | [73.9, 89.8] | [2.4, 18.6] | |
| ST5 | 60/82 (73.2) | 68/84 (81.0) | 0/43 (0.0) | <.0001 |
| | [62.7, 81.6] | [71.3, 87.9] | [0.0, 8.2] | |
| ST6 | 50/82 (61.0) | 60/84 (71.4) | 0/43 (0.0) | <.0001 |
| | [50.2, 70.8] | [61.0, 80.0] | [0.0, 8.2] | |
| ST1 to ST6 | 39/82 (47.6) | 54/84 (64.3) | 0/43 (0.0) | <.0001 |
| combined | [37.1, 58.2] | [53.6, 73.7] | [0.0, 8.2] | |
| ST1 and ST2 | 48/82 (58.5) | 58/84 (69.0) | 0/43 (0.0) | <.0001 |
| combined | [47.7, 68.6] | [58.5, 77.9] | [0.0, 8.2] | |

Initial data analysis including Day 208 (i.e. 1 month after the third vaccination) immunogenicity and safety data reveal that antibody titers further increased using a broader immunization schedule (schedule Month 0-2-6). Peak antibody levels (GMTs) at Day 208 in the 135 μg dose group ranged from 276.4 [ST1] to 539.0 [ST2] and in the 180 μg dose group ranged from 274.7 [ST1] to 596.8 [ST3] (see Table 5), which is a 1,7 to 2,7-fold increase in titers compared with respective titers obtained with the same treatment groups using a vaccination schedule of Month 0-1-2 (see Table 6). In line with ELISA data, GMTs for SBA titers also increased by factor 1,4 to 3,1 using the Month 0-2-6 schedule, compared with the M 0-1-2 schedule (Table 14). Table 5 provides an overview of Geometric Mean Titers (GMTs) for the second Phase 2 study and to Table 6 for comparison of GMTs using different vaccination schedules.

TABLE 5
Day 208 GMT (ELISA), second Phase 2 study (schedule Month 0-2-6), PP population

| | 135 μg | | | 180 μg | | |
|---|---|---|---|---|---|---|
| | 18-49 years | 50-65 years | 18-65 years | 18-49 years | 50-65 years | 18-65 years |
| | N = 55 | N = 29 | N = 84 | N = 57 | N = 29 | N = 86 |
| | [EU/mL] | [EU/mL] | [EU/mL] | [EU/mL] | [EU/mL] | [EU/mL] |
| ST1 | 284.1 | 263.5 | 276.4 | 291.2 | 246.6 | 274.7 |
| ST2 | 551.9 | 517.2 | 539.0 | 581.3 | 577.0 | 579.8 |
| ST3 | 573.5 | 434.5 | 518.4 | 633.4 | 534.3 | 596.8 |
| ST4 | 365.2 | 275.6 | 329.7 | 393.3 | 324.8 | 367.8 |
| ST5 | 358.8 | 287.0 | 330.8 | 371.0 | 337.2 | 358.8 |
| ST6 | 362.4 | 300.3 | 338.4 | 337.5 | 361.4 | 345.7 |

As depicted in Table 5, the immunological response in older adults, one of the main target groups for a Lyme vaccine, is particularly encouraging. Also, results did not indicate that prior exposure to Lyme (sero-positivity) has an impact on immunogenicity or safety (data not shown).

TABLE 6
Comparison of GMTs (ELISA) using different immunization schedules

| | Study 1 | | Study 2 | | | |
|---|---|---|---|---|---|---|
| | Schedule Month 0-1-2 | | Schedule Month 0-2-6 | | | |
| | Peak titers Day 85 | | Peak titers Day 208 | | | |
| | 135 μg | 180 μg | 135 μg | 180 μg | Ratio GMT Study 2 | |
| | 18-65 years | 18-65 years | 18-65 years | 18-65 years | (Day 208)/Study 1 | |
| | N = 188 | N = 189 | N = 84 | N = 86 | (Day 85) | |
| | [EU/mL] | [EU/mL] | [EU/mL] | [EU/mL] | 135 μg | 180 μg |
| ST1 | 101.1 | 115.8 | 276.4 | 274.7 | 2.7 | 2.4 |
| ST2 | 276.9 | 303.7 | 539.0 | 579.8 | 1.9 | 1.9 |
| ST3 | 282.2 | 308.6 | 518.4 | 596.8 | 1.8 | 1.9 |
| ST4 | 169.7 | 190.7 | 329.7 | 367.8 | 1.9 | 1.9 |
| ST5 | 174.6 | 199.6 | 330.8 | 358.8 | 1.9 | 1.8 |
| ST6 | 182.6 | 208.7 | 338.4 | 345.7 | 1.9 | 1.7 |

As in the first Phase 2 study older adults (age group 50-65 years) which are one of the high risk groups for acquiring Lyme disease have also shown an encouraging immune response, without statistical significant differences in GMTs between the two age groups (Table 5).

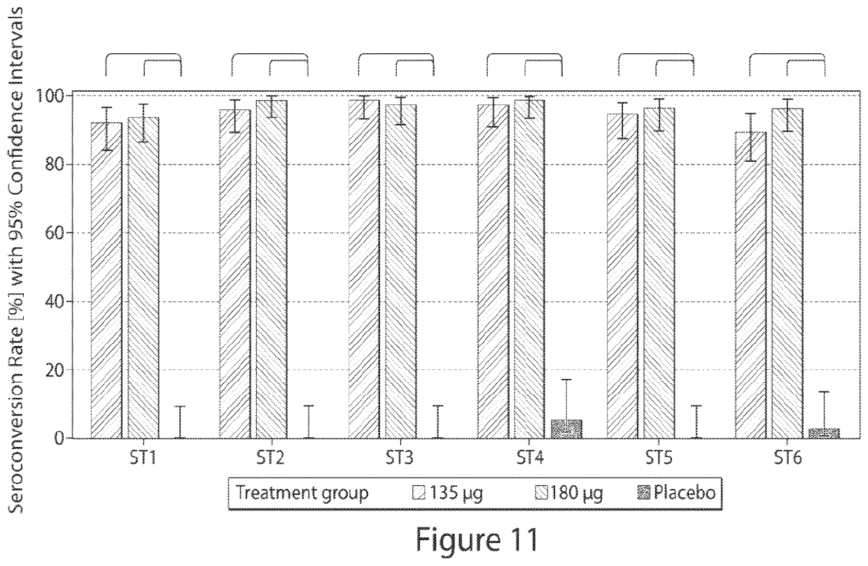

Seroconversion rates (SCRs) at Day 208 are depicted in Table 7 and FIG. 11. In the 135 μg dose, SCRs ranged from 89.6% (ST6) to 98.7 (ST3) and in the 180 μg group SCRs ranged from 93.8% (ST1) to 98.8% (ST2, ST4).

TABLE 7
Day 208 SCRs (ELISA) second Phase 2 study
(schedule Month 0-2-6), PP population

| | 135 μg | 180 μg |
|---|---|---|
| | N = 84 | N = 86 |
| | (%) | (%) |
| ST1 | 92.2 | 93.8 |
| ST2 | 96.1 | 98.8 |
| ST3 | 98.7 | 97.5 |
| ST4 | 97.4 | 98.8 |
| ST5 | 94.8 | 96.3 |
| ST6 | 89.6 | 96.3 |

Functionality of antibodies has been demonstrated for all serotypes using a serum bactericidal assay (SBA-see Materials and Methods). GMTs at Day 208 are summarized in Table 8.

TABLE 8
GMTs for functional antibodies as measured by SBA on Day 208.

| | 135 μg | 180 μg |
|---|---|---|
| | N = 84 | N = 86 |
| | SBA Titer | SBA Titer |
| ST1 | 47.0 | 52.3 |
| ST2 | 45.8 | 38.0 |
| ST3 | 1200.8 | 1408.0 |
| ST4 | 44.6 | 51.4 |
| ST5 | 31.4 | 36.8 |
| ST6 | 196.8 | 251.1 |

ELISA titer correlated significantly with respective SBA titer for each OspA serotype and individual subject sera, as shown in FIGS. 9A-9F. Spearman correlation coefficients ranged from 0.6487, p-value <0.0001, for serotype 4 to 0.9131, p-value <0.0001, for serotype 3.

TABLE 14
Comparison of GMTs (SBA) using different immunization schedules

| | Study 1 | | Study 2 | | | |
|---|---|---|---|---|---|---|
| | Schedule Month 0-1-2 | | Schedule Month 0-2-6 | | | |
| | Titers Day 85 | | Titers Day 208 | | | |
| | 135 μg | 180 μg | 135 μg | 180 μg | Ratio GMT Study 2 | |
| | 18-65 years | 18-65 years | 18-65 years | 18-65 years | (Day 208)/Study 1 | |
| | N = 188 | N = 189 | N = 84 | N = 86 | (Day 85) | |
| | [EU/mL] | [EU/mL] | [EU/mL] | [EU/mL] | 135 μg | 180 μg |
| ST1 | 17.5 | 21.4 | 47.0 | 52.3 | 2.7 | 2.4 |
| ST2 | 14.9 | 17.6 | 45.8 | 38.0 | 3.1 | 2.2 |
| ST3 | 737.6 | 700.1 | 1200.8 | 1408.0 | 1.6 | 2.0 |
| ST4 | 21.9 | 25.8 | 44.6 | 51.4 | 2.0 | 2.0 |
| ST5 | 23.2 | 24.4 | 31.4 | 36.8 | 1.4 | 1.5 |
| ST6 | 100.2 | 152.4 | 196.8 | 251.1 | 2.0 | 1.6 |

The multimeric Borrelia vaccine (“MBV”) was generally safe across all dose and age groups tested. No related Serious Adverse Events (SAEs) were observed in this or any study. Reactogenicity decreased after the first vaccination. Overall, the tolerability profile including rates of fever was comparable to other lipidated recombinant vaccines or lipid-containing formulations. Table 9 provides a comparison of safety data between the Phase 2 studies with said vaccine as well as comparison to other lipidated recombinant vaccines or lipid-containing formulations.

TABLE 9
Comparison of solicited local and systemic Adverse Events with other
lipidated recombinant vaccines or lipid-containing formulations.

| | MBV | MBV | MBV | MBV | | | |
|---|---|---|---|---|---|---|---|
| | (schedule | (schedule | (schedule | (schedule | Trumenba¹ | Bexsero² | Lymerix³ |
| | M 0-1-2) | M 0-2-6) | M 0-1-2) | M 0-2-6) | (schedule | (schedule | (schedule |
| | 18-65 years | 18-65 years | 18-65 years | 18-65 years | M 0-2-6) | M 0-2) | M 0-1-12) |
| | 135 μg | 135 μg | 180 μg | 180 μg | 18-25 years | 10-25 years | 15-70 years |
| | N = 214 | N = 97 | N = 205 | N = 98 | N = 1823-2425 | N = 110-114 | N = 402 |
| | % of subjects | % of subjects | % of subjects | % of subjects | % of subjects | % of subjects | % of subjects |
| Pain | | | | | | | |
| after 1^{st} vacc. | 76.0 | 78.4 | 81.8 | 73.2 | 84.2 | 90 | 82 |
| after 2^{nd} vacc. | 66.3 | 72.8 | 74.2 | 68.1 | 79.3 | 83 | 76 |
| after 3^{rd} vacc. | 60.5 | 67.4 | 63.8 | 71.1 | 80.4 | not applicable | 83 |
| Redness | | | | | | | |
| after 1^{st} vacc. | 5.3^{A} | 6.3^{A} | 3.0^{A} | 8.3^{A} | 13.8^{A} | 50 | 22 |
| after 2^{nd} vacc. | 6.4 | 2.2 | 8.7 | 7.7 | 11.8 | 45 | 17 |
| after 3^{rd} vacc. | 3.6 | 4.7 | 6.2 | 6.7 | 17.1 | n/a | 25 |
| Swelling | | | | | | | |
| after 1^{st} vacc. | 6.8^{A} | 5.2^{A} | 7.4^{A} | 8.3^{A} | 15.5^{A} | not reported | 14 |
| after 2^{nd} vacc. | 7.3 | 1.1 | 9.6 | 8.6 | 14.0 | not reported | 11 |
| after 3^{rd} vacc. | 4.0 | 3.5 | 5.2 | 4.5 | 16.6 | not applicable | 19 |
| Fever | | | | | | | |
| (varying def.) | | | | | | | |
| after 1^{st} vacc. | 1.0 | 3.2 | 0.0 | 2.2 | 2.4 | 1 | 1.5 |
| after 2^{nd} vacc. | 1.0 | 3.4 | 1.5 | 0.0 | 1.2 | 5 | 1.0 |
| after 3^{rd} vacc. | 0.5 | 0.0 | 1.0 | 1.2 | 2.0 | n/a | 1.0 |
| Headache | | | | | | | |
| after 1^{st} vacc. | 20.2 | 30.9 | 27.1 | 28.9 | 43.9 | 33 | 21 |
| after 2^{nd} vacc. | 16.6 | 23.9 | 17.8 | 24.5 | 33.1 | 34 | 14 |
| after 3^{rd} vacc. | 14.4 | 17.4 | 17.0 | 19.1 | 32.5 | n/a | 20 |
| Myalgia | | | | | | | |
| after 1^{st} vacc. | 38.5 | 41.2 | 36.0 | 37.1 | 25.9 | 49 | n/a |
| after 2^{nd} vacc. | 26.0 | 29.3 | 24.9 | 29.0 | 15.6 | 48 | n/a |
| after 3^{rd} vacc. | 18.0 | 26.7 | 17.5 | 32.6 | 16.9 | n/a | n/a |
| Arthralgia | | | | | | | |
| after 1^{st} vacc. | 10.1 | 11.5 | 7.9 | 8.3 | 19.6 | 13 | 12 |
| after 2^{nd} vacc. | 9.8 | 13.0 | 7.1 | 10.6 | 15.1 | 16 | 11 |
| after 3^{rd} vacc. | 8.5 | 8.1 | 8.2 | 11.2 | 12.6 | n/a | 13 |

¹Trumenba Highlights of prescribing information September 2017,
²Bexsero Highlights of prescribing information October 2017,
³PI Lymerix (Rates of solicited AEs).
^{A}“Any” is defined as the cumulative frequency of subjects who reported a reaction as “mild”, “moderate”, or “severe” within 7 days of vaccination. Mild (2.5-5.0 cm); moderate (>5.0-10.0 cm); severe (>10.0 cm).

In summary, clinical studies have shown that the multimeric Borrelia vaccine was generally safe across all doses and age groups tested. Immunogenicity was increased in Phase 2 using higher vaccine doses (135 μg, 180 μg) and an alternative immunization schedule (Month 0-2-6).

### Example 4: Phase 2 Clinical Trial with Multivalent Borrelia Vaccine Investigating Three-Dose or Two-Dose Primary Immunization Schedule of the Vaccine, and Booster Dose, in a Study Population Aged 5 to 65 Years

This study is a randomized, observer-blind, placebo controlled, multicenter Phase 2 study in healthy subjects aged 5 to 65 years. The study is conducted in two study parts (Part A: Main Study Phase, Part B: Booster Phase). The study is initiated with an age-descending enrollment of sentinel cohorts. Subject enrollment into Part A starts with the adult cohort that allows the generation and review of appropriate safety data before pediatric cohorts are initiated.

Subject Enrollment into Part A (Main Study Phase):

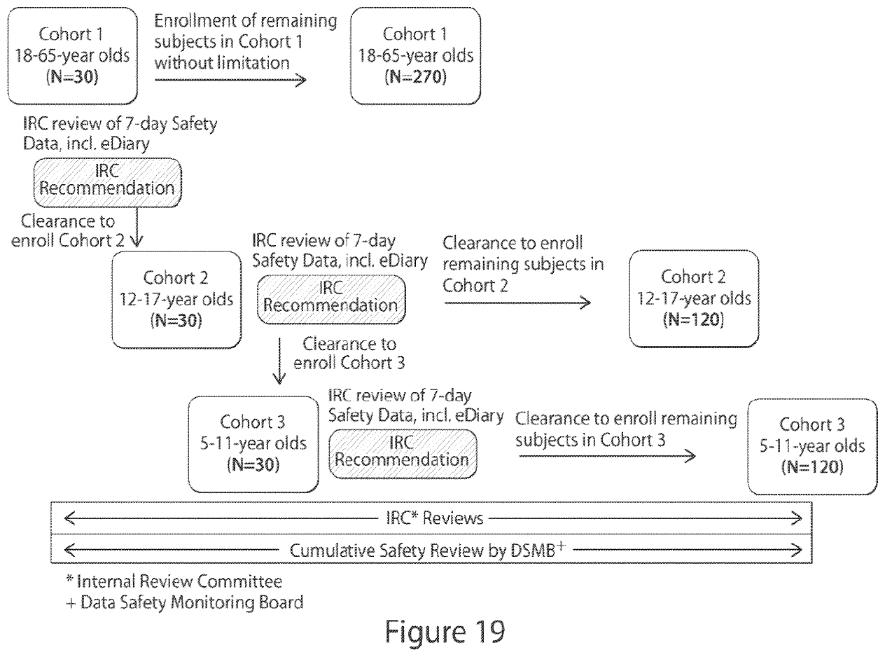

Enrollment is performed in an age-descending, staggered manner for the three age cohorts (see FIG. 19).

### Enrollment Cohort 1 (18-65 Years):

The enrollment starts with 30 sentinel adult subjects aged 18-65 years (Cohort 1). Subjects are randomized 1:1:1 into one of three study groups to receive a vaccination of the multimeric Borrelia vaccine (“MBV”) adjuvanted with aluminium hydroxide (Groups 1 and 2) or placebo (Group 3). After these 30 sentinel subjects have received the first vaccination and have completed a 7-day post vaccination safety follow up visit (Visit 1A) including review of 7-day eDiary data, safety data is reviewed in an unblinded manner by an independent Internal Review Committee (IRC). Based on this data, the IRC makes a recommendation on whether enrollment of the adolescent age cohort (12-17 years, Cohort 2) can be initiated. Enrollment of the remaining 270 adult subjects of Cohort 1 continues without limitations during the IRC review.

Enrollment Cohort 2 (12-17 years):

Upon IRC clearance, 30 sentinel adolescent subjects aged 12-17 years (Cohort 2) are enrolled. Subjects are randomized 1:1:1 into one of the three study groups to receive a vaccination of the multimeric Borrelia vaccine adjuvanted with aluminium hydroxide (Groups 1 and 2) or placebo (Group 3). Again, after these subjects have completed their safety follow up visit 7 days after the first vaccination (Visit 1A) including review of 7-day eDiary data, safety data is reviewed by the IRC. The IRC recommends whether enrollment of the youngest age cohort (5-11 years of age) can be initiated. In this age cohort, enrollment of the remaining 120 adolescent subjects is only initiated after IRC clearance.

Enrollment Cohort 3 (5-11 years):

Upon IRC clearance, 30 sentinel subjects aged 5-11 years (Cohort 3) are enrolled. Subjects are randomized 1:1:1 into one of the three study groups to receive a vaccination of the multimeric Borrelia vaccine adjuvanted with aluminium hydroxide (Groups 1 and 2) or placebo (Group 3). Again, after these 30 subjects have completed their safety follow up visit 7 days after the first vaccination (Visit 1A) including review of 7-day eDiary data, safety data is reviewed by the IRC. Enrollment of the remaining 120 subjects in this age cohort is only be initiated after IRC clearance. An external independent Data Safety Monitoring Board (DSMB) reviews accruing safety data in an unblinded manner at regular intervals and might recommend adjusting, pausing or discontinuing the study at any time.

Subject Enrollment into Part B (Booster Phase):
Multimeric Borrelia vaccine Booster (Group 1 or 2)

All eligible subjects (max. 200; see Table 15) within the study group (Group 1 or Group 2), who received all immunizations according to the primary immunization schedule that is selected for further development, continue in Part B, and receive an additional MBV vaccination at Month 18.

### Placebo Booster (Group 1 or 2)

Approximately half of the subjects (i.e. max. 100, maintaining the 2:1:1 age stratification; see Table 15) within the study group (Group 1 or Group 2) who received all immunizations according to the alternative primary schedule (i.e., the schedule that is not selected for further development) and who are eligible for the Booster Phase, continue in Part B. For this purpose, the first 50 eligible adult subjects (age group 18-65 years), the first 25 eligible adolescent subjects (age group 12-17) and the first 25 eligible subjects (age group 5-11 years) are included in the booster phase as they present for their Visit 8/Month 18 visit. These subjects receive a placebo injection at Month 18. Remaining subjects from this study group are discontinued.

### Placebo Booster (Group 3)

Approximately half of the subjects (i.e. max. 100, maintaining the 2:1:1 age stratification; see Table 15) within the placebo study group (Group 3), who received all injections according to schedule and who are eligible for the Booster Phase, continue in Part B. For this purpose, the first 50 eligible adult subjects (age group 18-65 years), the first 25 eligible adolescent subjects (age group 12-17) and the first 25 eligible subjects (age group 5-11 years) are included in the booster phase as they present for their Visit 8/Month 18 visit. These subjects receive an additional placebo injection at Month 18. Remaining subjects from this study group are discontinued. The study sponsor and study statisticians are unblinded at the time of the primary endpoint analysis, i.e. prior to subject enrollment into Part B. Through an electronic system the study site is notified once the number of registered eligible subjects is reached for Part B.

### Study Design

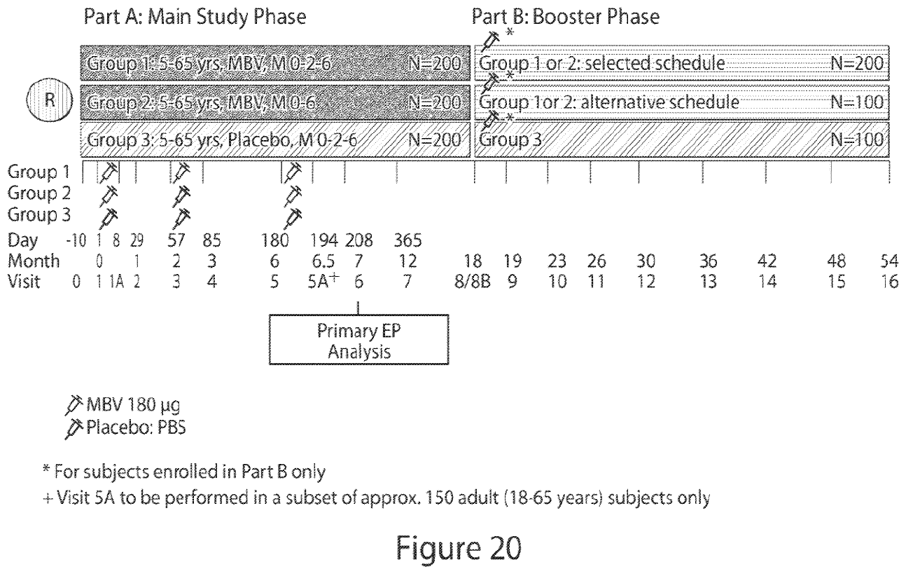

The study is conducted in two study parts (Part A: Main Study Phase, Part B: Booster Phase). Refer to FIG. 20 for the study design.

In Part A (Main Study Phase) a total of approximately 600 subjects aged 5 to 65 years is randomized 1:1:1 into three study groups: Group 1 (approximately 200 subjects) receive three vaccinations of the multimeric Borrelia vaccine adjuvanted with aluminium hydroxide at Month 0-2-6. Group 2 (approximately 200 subjects) receive two vaccinations of the multimeric Borrelia vaccine adjuvanted with aluminium hydroxide at Month 0-6 and a placebo injection at Month 2 in order to keep the blind. Group 3 (approximately 200 subjects) receive three placebo injections at Month 0-2-6. Within each study group subjects are enrolled 2:1:1 in three age cohorts (18-65 years, 12-17 years and 5-11 years).

In Part A, all subjects receive three I.M. injections of either MBV or placebo at Month 0-2-6 (i.e. Day 1-57-180). On Day 8/Visit 1A (i.e. 7 days after the first vaccination) a safety visit is performed (phone call for subjects aged 18-65, in-person visit for subjects aged 5-17 years). In-person visits are scheduled for all age cohorts one month after each vaccination. Blood samples for immunogenicity assessments are collected at the screening visit, Day 85, Day 180, Day 194 (in a subset of adult subjects), Day 208, Day 365/Month 12 and at Month 18.

Based on safety and immunogenicity data from Day 208/Visit 6 of Part A (i.e. 1 month after the third immunization), data is used to guide the decision for Part B booster.

In Part B (Booster Phase) eligible subjects from the Group that received selected primary schedule in Part A (either Group 1 or 2) receive a booster dose of the multimeric Borrelia vaccine adjuvanted with aluminium hydroxide at Month 18.

For safety comparison, placebo injections are administered to approximately 100 subjects, who were enrolled in the group that was vaccinated in Part A according to the alternative vaccination schedule, (i.e. either Group 1 or 2 which received vaccinations according to the primary immunization schedule that is not selected for further vaccine development), and to approximately 100 subjects of Group 3. It is targeted to maintain the 2:1:1 age stratification (18-65 years, 12-17 years and 5-11 years) in all study groups. All subjects involved in Part B are followed-up for further 3 years (i.e. up to Month 54) with study visits at Months 19, 23, 26, 30, 36, 42, 48 and 54.

Table 15 shows an overview of study groups and treatments. Treatments administered have an injection volume of 0.5 ml.

TABLE 15
Study Groups and Vaccinations

| | | | Age | | |
|---|---|---|---|---|---|
| | Study | | in | | Vaccination |
| | Group | Subjects | years | Treatment | Schedule |
| Part A: | Group 1 | Total: 200 | | MBV 180 μg | Month 0-2-6 |
| Main | | 100 | 18-65 | | |
| Study | | 50 | 12-17 | | |
| Phase | | 50 | 5-11 | | |
| | Group 2 | Total: 200 | | MBV 180 μg | Month 0-6 |
| | | 100 | 18-65 | Placebo | Month 2* |
| | | 50 | 12-17 | | |
| | | 50 | 5-11 | | |
| | Group 3 | Total: 200 | | Placebo | Month 0-2-6 |
| | | 100 | 18-65 | | |
| | | 50 | 12-17 | | |
| | | 50 | 5-11 | | |
| Part B: | Group 1 or 2: | Total: 200 | | MBV 180 μg | Month 18 |
| Booster | Selected | 100 | 18-65 | | |
| Phase | Schedule | 50 | 12-17 | | |
| | | 50 | 5-11 | | |
| | Group 1 or 2: | Total: 100 | | Placebo | Month 18 |
| | Alternative | 50 | 18-65 | | |
| | Schedule | 25 | 12-17 | | |
| | | 25 | 5-11 | | |
| | Group 3 | Total: 100 | | Placebo | Month 18 |
| | | 50 | 18-65 | | |
| | | 25 | 12-17 | | |
| | | 25 | 5-11 | | |

*In order to keep the blind, subjects assigned to Group 2 will receive a placebo at Month 2.

1.-66. (canceled)

67. A composition comprising:
(a) a first fusion protein comprising the amino acid sequence of SEQ ID NO: 1 (Lip-S1D1-S2D1);
(b) a second fusion protein comprising the amino acid sequence of SEQ ID NO: 2 (Lip-S4D1-S3hybD1); and
(c) a third fusion protein comprising the amino acid sequence of SEQ ID NO: 3 (Lip-S5D1-S6D1),
wherein the composition comprises two or more doses,
wherein:
(1) each dose of the composition comprises a total protein content of the three fusion proteins of 135 to 200 μg for administration to a human adult; or
(2) each dose of the composition comprises a total protein content of the three fusion proteins of 60 to 100 μg for administration to a human child,
wherein each dose of the composition comprises the first, second, and third fusion proteins in a weight ratio of 1:2:1, 1:3:1, 1:1:2, 1:1:3, 1:2:2, 1:2:3, 1:3:2, 1:3:3, 2:1:1, 2:1:2, 2:1:3, 2:2:3, 2:2:1, 2:3:1, 2:3:2, 2:3:3, 3:1:1, 3:1:2, 3:1:3, 3:2:1, 3:2:2, 3:2:3, 3:3:1, or 3:3:2.

68. The composition of claim 67, wherein each dose comprises, in a volume of 0.5 mL, between 5 mM and 50 mM sodium phosphate, between 100 and 200 mM sodium chloride, between 5 mM and 25 mM L-methionine, between 2.5% and 10% sucrose, between 0.01% and 0.1% polysorbate 20 and between 0.1% and 0.2% (w/v) aluminium hydroxide.

69. The composition of claim 67, wherein each dose comprises, in a volume of 0.5 mL, 10 mM sodium phosphate, 150 mM sodium chloride, 10 mM L-methionine, 5% sucrose, 0.05% polysorbate 20, and 0.15% (w/v) aluminium hydroxide at pH 6.7±0.2.

70. The composition of claim 67, wherein the three fusion proteins comprise at least 80% of all proteins in each dose.

71. The composition of claim 67, wherein the composition comprises:
a primary immunization series of doses comprising at least two or three doses; and
two or more booster doses for administration every year after the last primary immunization or at the beginning of each tick season.

72. The composition of claim 67, wherein each dose of the composition comprises:
a total protein content of the three fusion proteins of 135 μg or 180 μg for administration to a human adult, or
a total protein content of the three fusion proteins of 67.5 μg or 90 μg for administration to a human child.

73. The composition of claim 67, wherein administration of the composition induces an immune response comprising an anti-OspA serotype 1, an anti-OspA serotype 2, an anti-OspA serotype 3, an anti-OspA serotype 4, an anti-OspA serotype 5, and an anti-OspA serotype 6 antibody response with bactericidal activity.

74. A composition comprising:
(a) a first fusion protein comprising the amino acid sequence of SEQ ID NO: 1 (Lip-S1D1-S2D1);
(b) a second fusion protein comprising the amino acid sequence of SEQ ID NO: 2 (Lip-S4D1-S3hybD1); and
(c) a third fusion protein comprising the amino acid sequence of SEQ ID NO: 3 (Lip-S5D1-S6D1),
wherein the composition comprises two or more doses, each dose comprising, in a volume of 0.5 mL:
the three fusion proteins at a 1:1:1 weight ratio, between 5 mM and 50 mM sodium phosphate, between 100 and 200 mM sodium chloride, between 5 mM and 25 mM L-methionine, between 2.5% and 10% sucrose, between 0.01% and 0.1% polysorbate 20 and between 0.1% and 0.2% (w/v) aluminium hydroxide,
and wherein:
(1) each dose of the composition comprises a total protein content of the three fusion proteins of 135 to 200 μg for administration to a human adult; or
(2) each dose of the composition comprises a total protein content of the three fusion proteins of 60 to 100 μg for administration to a human child.

75. The composition of claim 74, wherein each dose comprises, in a volume of 0.5 mL, 10 mM sodium phosphate, 150 mM sodium chloride, 10 mM L-methionine, 5% sucrose, 0.05% polysorbate 20, and 0.15% (w/v) aluminium hydroxide at pH 6.7±0.2.

76. The composition of claim 74, wherein the three fusion proteins comprise at least 80% of all proteins in each dose.

77. The composition of claim 74, wherein the composition comprises:
a primary immunization series of doses comprising at least two or three doses; and
two or more booster doses for administration every year after the last primary immunization or at the beginning of each tick season.

78. The composition of claim 74, wherein each dose of the composition comprises:
a total protein content of the three fusion proteins of 135 μg or 180 μg for administration to a human adult, or
a total protein content of the three fusion proteins of 67.5 μg or 90 μg for administration to a human child.

79. The composition of claim 74, wherein administration of the composition induces an immune response comprising an anti-OspA serotype 1, an anti-OspA serotype 2, an anti-OspA serotype 3, an anti-OspA serotype 4, an anti-OspA serotype 5, and an anti-OspA serotype 6 antibody response with bactericidal activity.

80. A method of eliciting an immune response against Lyme disease in a human adult or human child, comprising administering to the human adult or human child a primary immunization series and two or more booster doses of a composition comprising:
(a) a first fusion protein comprising the amino acid sequence of SEQ ID NO: 1 (Lip-S1D1-S2D1),
(b) a second fusion protein comprising the amino acid sequence of SEQ ID NO: 2 (Lip-S4D1-S3hybD1), and
(c) a third fusion protein comprising the amino acid sequence of SEQ ID NO: 3 (Lip-S5D1-S6D1),
wherein each dose of the composition comprises, in a volume of 0.5 mL, the three fusion proteins at a 1:1:1 weight ratio,
wherein:
(1) each dose of the composition comprises a total protein content of the three fusion proteins of 135 to 200 μg when administered to the human adult; or
(2) each dose of the composition comprises a total protein content of the three fusion proteins of 60 to 100 μg when administered to the human child,
wherein the primary immunization series comprises administering at least two or at least three doses of the composition, and the two or more booster doses are administered every year after the last primary immunization or at the beginning of each tick season.

81. The method of claim 80, wherein each dose comprises, in a volume of 0.5 mL, between 5 mM and 50 mM sodium phosphate, between 100 and 200 mM sodium chloride, between 5 mM and 25 mM L-methionine, between 2.5% and 10% sucrose, between 0.01% and 0.1% polysorbate 20 and between 0.1% and 0.2% (w/v) aluminium hydroxide.

82. The method of claim 80, wherein each dose comprises, in a volume of 0.5 mL, 10 mM sodium phosphate, 150 mM sodium chloride, 10 mM L-methionine, 5% sucrose, 0.05% polysorbate 20, and 0.15% (w/v) aluminium hydroxide at pH 6.7±0.2.

83. The method of claim 80, wherein the three fusion proteins comprise at least 80% of all proteins in each dose.

84. The method of claim 80, wherein each dose of the composition comprises:
a total protein content of the three fusion proteins of 135 μg or 180 μg for administration to a human adult, or
a total protein content of the three fusion proteins of 67.5 μg or 90 μg for administration to a human child.

85. The method of claim 80, wherein the immune response comprises an anti-OspA serotype 1, an anti-OspA serotype 2, an anti-OspA serotype 3, an anti-OspA serotype 4, an anti-OspA serotype 5, and an anti-OspA serotype 6 antibody response with bactericidal activity.

86. The method of claim 80, wherein the primary immunization series comprises administering at least three doses, wherein the second dose is administered at least 4 weeks to at most 3 months after the first dose, and the third dose is administered at least 5 months to at most 7 months after the first dose.
